(19) United States
(12) Patent Application Publication
(10) Pub. No.: US 2011/0098623 A1
(43) Pub. Date: Apr. 28, 2011

(54) DEVICE AND METHOD OF USING SUPERPARAMAGNETIC NANOPARTICLES IN TREATMENT AND REMOVAL OF CELLS

(75) Inventors: Zhongju John Zhang, Alpharetta, GA (US); Kenneth Edward Scarberry, Clearwater, FL (US); Erin Beth Dickerson, Atlanta, GA (US); John Francis McDonald, Arnoldsville, GA (US)

(73) Assignee: Georgia Tech Research Corporation

(21) Appl. No.: 12/999,077

(22) PCT Filed: Jun. 17, 2009
(86) PCT No.: PCT/US09/47717

§ 371 Date: Dec. 15, 2010

Related U.S. Application Data
(60) Provisional application No. 61/073,161, filed on Jun. 17, 2008.
(60) Provisional application No. 61/073,973, filed on Jun. 19, 2008.

(51) Int. Cl. A61M 1/34; A61F 2/01; B01D 35/06; B03C 1/02; B82Y 5/00

(52) U.S. Cl. 604/5.02; 604/6.09; 604/93.01; 210/222; 210/175; 210/198.1; 210/205; 977/838; 977/773; 977/908

(57) ABSTRACT

Methods and devices for selectively removing from a subject a target cell, pathogen, or virus expressing a binding partner on its surface are presented. In one embodiment, the device contains an excorporeal circuit, which includes, at least, a magnetic filter comprising a magnet and a removable, magnetizable substrate capable of capturing magnetic nanomaterials; and a pump in fluid communication with the magnetic filter, wherein the pump moves fluid through the excorporeal circuit. The magnet is capable of generating a magnetic field sufficient to capture magnetic nanomaterials in the magnetic field. In a preferred embodiment, the target cells are cancer cells and/or cells infected with pathogenic agents. The devices may be designed for extracorporeal or in vivo uses. Functionalized superparamagnetic nanoparticles are either mixed ex vivo with a biological fluid from the patient or injected into the patient. Then the biological fluid, which includes the nanoparticles is transported to the magnetic filter to remove any nanoparticles that are complexed to the target cells, pathogens, or virus, and any free nanoparticles. Optionally, the functionalized nanoparticles contain and deliver a therapeutic agent. In one embodiment, the therapeutic agent is released when the nanoparticle binds to the target cells, pathogens, or virus.

### CROSS-REFERENCE TO RELATED APPLICATION

This application claims priority to U.S. Provisional application, U.S. Ser. No. 61/073,161, filed Jun. 17, 2008 and U.S. Provisional application, U.S. Ser. No. 61/073,973, filed Jun. 19, 2008.

### FIELD OF THE INVENTION

This invention is in the field of devices and methods for using and removing magnetic nanoparticles, particularly for the treatment of cancer.

### BACKGROUND OF THE INVENTION

Cancer of all types is the second leading cause of death in the United States. The morbidity of most cancers is often related to the metastatic dissemination of cells sloughing off the primary tumor. Although improved chemical and radiation therapies are resulting in less collateral damage to healthy cells, there remains a large market for targeted modalities. Long-term survival rates could be extended significantly if metastatic cells or the cells remaining after surgical excision of the tumor mass could be more effectively removed from the patient.

Abdominal cancer accounts for 40% of more than 10 million people currently living with cancer, and there are more than 250,000 newly diagnosed abdominal cancers each year, many of which are detected in later stages where surgery is a preferred and recommended therapeutic option, especially when the tumor is surgically accessible and the risk of collateral damage from the procedure is low. However, often during the surgical procedure, malignant cells will have broken free and be left in the peritoneal cavity. Following surgical extraction of the tumor, chemotherapy is typically recommended to kill the residual malignant cells but chemotherapy is not completely effective.

Most ovarian cancer recurrence is the result of the metastasis of free-floating cancer cells to secondary regions in the abdomen (omentum, liver, etc). The prognosis and therapies required to treat ovarian and other cancers following surgery could improve significantly if cells remaining after surgical excision of the tumor mass could be more effectively removed from the patient.

Many shortcomings are associated with currently available therapies for cancer. There is a need for a therapeutic approach that can improve targetability, reduce collateral damage to healthy cells, and ultimately improve the long-term prognosis for the patient.

Another area that has not received much consideration is the elimination of the nanoparticles from the body after their therapeutic tour is complete. Currently, most of the suggested procedures are surgical in nature and highly invasive. Minimally invasive techniques could increase social acceptance of using nanoparticles in biomedical procedures.

Therefore it is an object of the invention to provide improved devices and methods for removing cancer cells from a patient.

It is a further object of the invention to provide improved methods for removing nanoparticles from a patient.

It is a further object of the invention to provide methods for improving the treatment of and/or removal of cancer in a patient.

### SUMMARY OF THE INVENTION

Methods and devices for selectively removing from a subject a target cell, pathogen, or virus expressing a binding partner on its surface are presented. In one embodiment, the device contains an excorporeal circuit, which includes, at least, a magnetic filter comprising a magnet and a removable, magnetizable substrate capable of capturing magnetic nanomaterials; and a pump in fluid communication with the magnetic filter, wherein the pump moves fluid through the excorporeal circuit. The magnet is capable of generating a magnetic field sufficient to capture magnetic nanomaterials in the magnetic field. In a preferred embodiment, the target cells are cancer cells and/or cells infected with pathogenic agents. The devices may be designed for extracorporeal or in vivo uses. Functionalized superparamagnetic nanoparticles are either mixed ex viva with a biological fluid from the patient or injected into the patient. Then the biological fluid, which includes the nanoparticles is transported to the magnetic filter to remove any nanoparticles that are complexed to the target cells, pathogens, or virus, and any free nanoparticles. Optionally, the functionalized nanoparticles contain and deliver a therapeutic agent. In one embodiment, the therapeutic agent is released when the nanoparticle binds to the target cells, pathogens, or virus.

### BRIEF DESCRIPTION OF THE DRAWINGS

FIGS. 1A and 1B are schematics of exemplary devices for ex vivo treatment of a biofluid with superparamagnetic nanoparticles.

FIG. 2A-C are schematics of an exemplary modifications to hemodialysis devices of in vivo treatment of a patient with superparamagnetic nanoparticles.

FIG. 3 is a bar graph of the extraction efficiencies of the Hey and BG-1 cells in the cell populations extracted from the peritoneum of three Balb/C female mice. The ratios were averaged from five counts performed on each of three mice. Error bars show the standard deviations.

FIGS. 4A-K are dot plots of flow cytometry analysis of ascites samples for patient 914. FIGS. 4A-C are dot plots for Trials 1-3, showing gated populations, frequencies, and population labels (1-4). FIGS. 4D-F are dot plots of flow cytometry analysis of filtrand samples for patient 914 (Trials 1-3) showing gates copied from untreated ascites trial (Trial 1), frequencies, and population labels (1-4). FIGS. 4G-I are dot plots of the filtrand extracted from patient samples (Patient 914) using nanoparticles having no peptide conjugates. Population gates were copied from previous trials performed on untreated ascites and pure nanoparticle samples. FIGS. 4J and K are dot plots of superparamagnetic nanoparticles coated with glucuronic acid with (FIG. 4J) and without (FIG. 4K) peptide functions showing gates copied from untreated ascites trial (Trial 1)

FIG. 5 is a schematic of the experimental set up used in Example 3.

FIG. 6 is a survival curve of the % of the mice in a particular group alive with the number of days following intraperitoneal injection for the Control A group (“Control”) (a straight line at day 36), the Control B group (“Treatment/No Nanoparticles”) (a stepwise line that ends at day 44) and the Experimental group (Treatment/With Nanoparticles) (a stepwise, line that ends at day 60).

FIG. 7 is a line graph of pg of p24/ml versus trial number of HIV-1 samples that are unchallenged, challenged with high concentration of superparamagnetic nanoparticles having no conjugates, challenged with low concentration superparamagnetic nanoparticles with low concentration of anti-cp120 conjugates, or challenged with high concentration of superparamagnetic nanoparticles with high concentration of anti-cp120 conjugates.

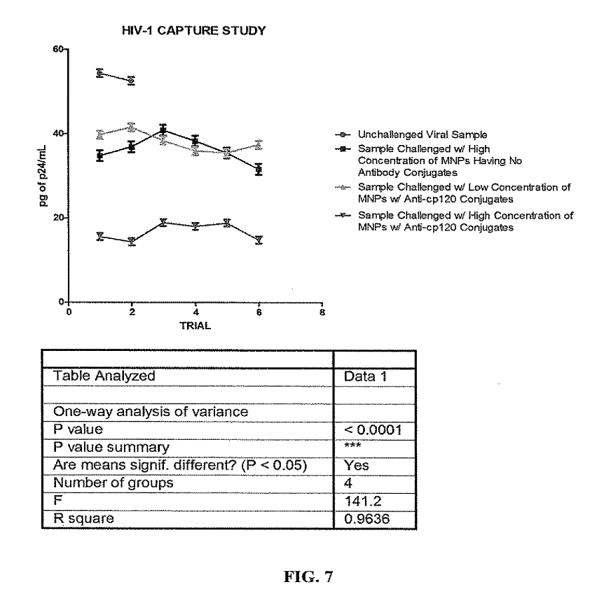

### DETAILED DESCRIPTION OF THE INVENTION

### I. Devices

A variety of different devices and systems that contain a magnetic filter/trap can be used to treat a patient in vivo or ex vivo with superparamagnetic nanoparticles designed to bind with a target cell, pathogen, or virus expressing a binding partner on its surface. In one embodiment, the target cells are diseased cells expressing a specific surface membrane binding partner. In one embodiment, the nanoparticles are functionalized with one or more molecules designed to specifically bind with one or more cancer cells.

In one embodiment the device contains an excorporeal circuit, wherein the excorporeal circuit contains a magnetic filter comprising a magnet capable of generating a magnetic field sufficient to capture magnetic nanomaterials in the magnetic field and a removable, magnetizable substrate capable of capturing magnetic nanomaterials; and a pump in fluid communication with the magnetic filter, wherein the pump moves fluid through the excorporeal circuit.

In another embodiment, one or more magnetic filter(s) are combined with existing devices to capture magnetic nanoparticles.

Preferably all of the elements of the device that contact the biofluid are sterilizable. Although the tubing used throughout the device may be sterilizable, in some embodiments, typically, in use the tubing may be disposed and therefore, it is not a requirement that the tubing be sterilizable.

As used herein “biological fluid” or “biofluid” refers to any fluid from a mammal, typically a human. Biological fluids include but are not limited to blood, blood serum, plasma, cerebrospinal fluid, lymph, and peritoneal fluid.

A. Magnetic Filter

The magnetic filter (10) can have any shape or size suitable for attracting the magnetic, paramagnetic, and/or superparamagnetic nanoparticles and materials attached thereto.

The magnetic filter (10) contains at least one magnet (12), and preferably also contains a magnetizable substrate (14), and optionally contains a vessel (16).

In one embodiment, the magnetic filter does not contain a magnetizable substrate. In this embodiment, the magnetic filter contains a vessel (16) through which the nanoparticles pass and one or more magnets (12) attached to at least a portion of the wall of the vessel. The magnets may be on the outer surface of the wall of the vessel or may be on the inner surface of the vessel (i.e. the side in contact with the biofluid containing the nanoparticles.

a. Magnet

The magnetic filter (10) contains a magnet (12), such as permanent magnet or an electromagnet. The magnet is capable of generating a magnetic field sufficient to capture magnetic nanomaterials in the magnetic field.

Typically the magnet provides a magnetic flux field of at least about 500 gauss measured at the point of contact between the superparamagnetic nanoparticle and the magnet or magnetizable material. Although magnets with greater magnetic flux fields, such as 1,500 gauss or greater, or 2,500 gauss or greater, may be used, they are not typically required.

In one embodiment the magnetic filter contains an external magnet (12) which produces a magnetic field and a screen (14) formed of a magnetically attractive material. In another embodiment, the screen and the magnet are one component.

As used herein “magnet” refers to a material that both produces its own magnetic field and responds to magnetic fields. Magnets include permanent magnets, which remain magnetized, and impermanent magnets, which lose their memory of previous magnetizations.

As used herein “magnetically attractive material” refers to materials that do not produce a magnetic field, but are attracted to a magnetic field or to each other when in the presence of a magnetic field, and include paramagnetic materials. Magnetically attractive materials include but are not limited to the following materials: iron, preferably iron coated with Teflon, polyimide, or parylene, or another suitable material to make it biocompatible, and steel.

i. Permanent Magnet

Any permanent magnet may be included in the magnetic filter. Suitable permanent magnets include ferromagnetic and ferrimagnetic materials. Magnets include but are not limited to the following materials: Neodymium (Rare Earth), Samarium Cobalt (Rare Earth), Ceramic (Ferrite), and Alnico (Aluminum Nickel Cobalt).

ii. Electromagnet

In one embodiment, the magnetic filter contains an electromagnet. In this embodiment, the electromagnet is preferably attached to a modular power supply via a tether. The tether, in addition to carrying the conductors, may provide conduits for water or gas circulation to cool the electromagnetic probe and leads. For safety purposes, the tether preferably has a braided ground wire. Thus, if the conductive leads carrying the high current to drive the electromagnet were in any way exposed, the tether would short to ground, and the power supply would be immediately shut down.

Benefits to using an electromagnet in place of a permanent magnet include, allowing the magnetic field to be further localized, resulting in a more focused and stronger magnetic field. Additionally, the electromagnet is switchable, so that it can be easily turned off when not it is in use.

Additionally, the electromagnet can serve as a monitor, since it can monitor and indicate the presence of another ferrous material that is being attracted to it. As such, a numeric or graphical indicator on the electromagnet power supply could provide information to the user regarding the presence and/or relative amounts of magnetic nanoparticles attracted to the electromagnet.

b. Screen

Preferably the screen (14) is formed from a sterilizable material, such as one which can be autoclaved. Preferably the magnetizable substrate capable of capturing magnetic nanomaterials is a screen.

Preferably the screen is removable from and replaceable in the vessel (16). This allows the filtered particles to be easily removed from the screen and tested without disassembling the rest of the device. Also, this allows for the cleaned filter to be replaced and reused with the same patient, or sterilized and reused with a different patient.

i. Shape

In one embodiment, the screen (14) contains a greater surface area than the magnet to provide maximum space for the magnetic nanoparticles to adhere to the surface of the screen.

In one embodiment, the screen is in the shape of a coil. In another embodiment, the screen has a rectangular cross section and is in the form of a mesh or lattice. In another embodiment, the screen contains a plurality of slats upon which the nanoparticles may attach.

ii. Materials

The screen is typically fowled of a magnetically attractive material, such iron or steel, preferably coated with a suitable coating material to make the screen biocompatible.

c. Vessel

The vessel (16) for the magnetic filter contains an inlet (17) and an outlet (18) and a suitable volume for the nanoparticles, the biocompatible suspending fluid and the biofluid, and is shaped to contain the screen, if one is used. In one embodiment, the vessel contains an attachment means, such as a slot, or clips (19a,b), for attaching the screen to the vessel. The vessel has a suitable volume to contain the required amount of nanoparticles and biofluid for a given treatment or round of treatment. Optionally, the vessel is graduated to indicate the volume of fluid inside the vessel. Optionally the vessel contains one or more sensors (20) to indicate the fluid level, temperature, or other properties of the material inside of the vessel. The sensors are in electrical communication or wireless communication with a processor. Preferably the processor is part of the management component (150).

Any suitable material may be used to form the vessel. Preferably the vessel is sterilizable. Typical materials include, glass, polyproplylene, polymethylpentene, and the like.

B. Extracorporeal Device for Ex Viva Treatment

In one embodiment, the device is designed to remove one or more fluids from a patient, add magnetic nanoparticles to the fluid (after the fluid is outside of the patient), and then remove the magnetic nanoparticles along with any materials coupled thereto, and finally return the processed biological fluid back to the patient.

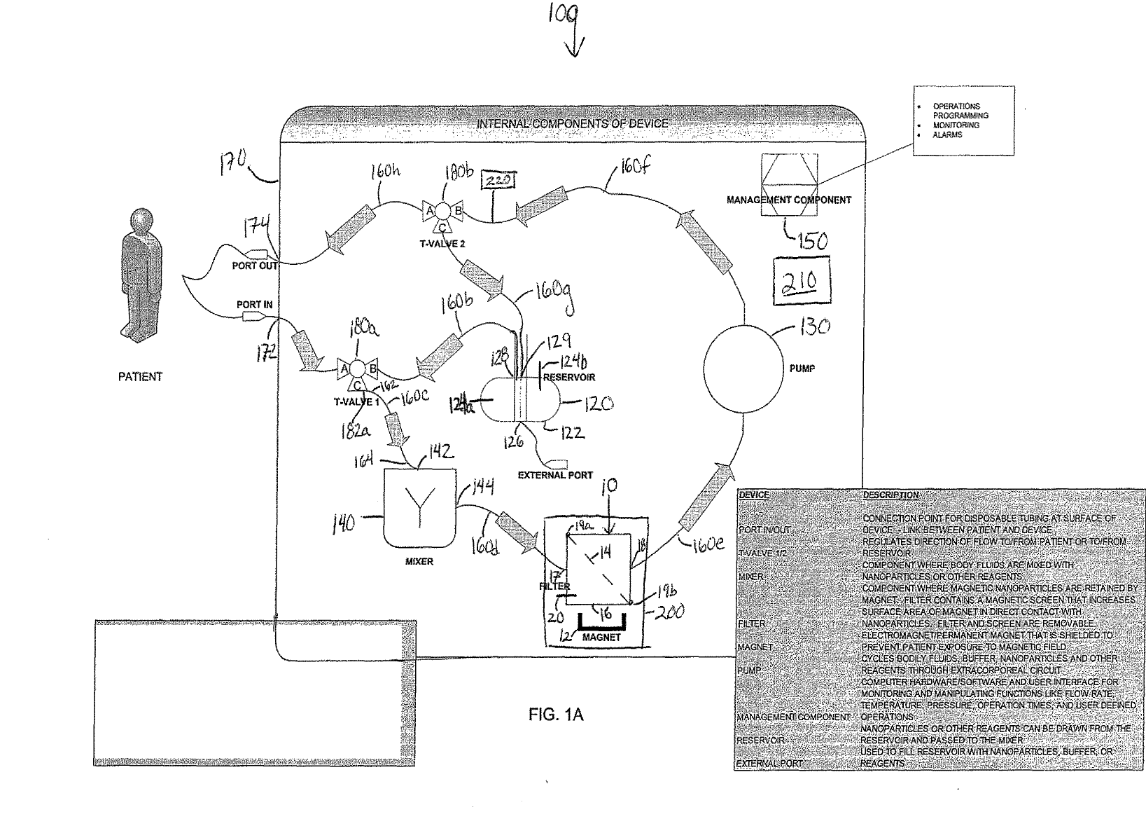

A variety of different configurations of the various components in the extracorporeal device may be used. Two exemplary configurations are illustrated in FIGS. 1A and 1B.

The extracorporeal device (100) typically contains the following components: a reservoir (120) for supplying the nanoparticles to the biological fluid, a magnetic filter (10), a pump (130), and tubing (160) connecting the various components of the device and allowing for the flow of the biofluid and/or nanoparticles through one or more elements of the device. Typically, the device also contains a mixing chamber (140) in which the nanoparticles and biological fluid are mixed prior to entry into the magnetic filter. Preferably, the device also contains at least one management component (150). Preferably these components are contained within a housing (180).

An exemplary device for selectively removing a target cell expressing a binding partner on the cell surface is illustrated in FIG. 1. It includes a reservoir having an inlet and an outlet. In use, the reservoir is typically filled with superparamagnetic particles functionalized with a binding partner that binds to the binding partner on the surface of the target cell. The device further includes a magnetic filter in fluid communication with the reservoir, wherein the magnetic filter has a magnet and screen capable of binding magnetic particles. Finally, the device includes a pump in fluid communication with the magnetic filter, wherein the pump moves fluid from the reservoir to the magnetic filter.

a. Reservoir

The reservoir (120) is designed to contain the nanoparticles and a biocompatible suspending fluid, optionally, the reservoir may be filed with a buffer or other reagents. The reservoir has a suitable volume to contain the required amount of nanoparticles for a given treatment or round of treatment. Any suitable vessel (122) may be used as a reservoir. Optionally, the vessel is graduated to indicate the volume of fluid inside the vessel. Optionally the vessel contains one or more sensors (124) to indicate the fluid level, temperature, or other properties of the material inside of the vessel. The sensors are in electrical communication or wireless communication with a processor. Preferably the processor is part of the management component (150).

Any suitable material may be used to form the reservoir. Preferably the reservoir is sterilizable. The reservoir is formed from a glass, polyproplylene, polymethylpentene, and the like.

The reservoir has an inlet (126) through which the nanoparticles, suspending agent, buffer, and/or other fluid enter the reservoir. The reservoir also contains an outlet (128) through which the nanoparticles, suspending agent, buffer, and/or other fluid exit the reservoir. Optionally, the reservoir contains a recycle inlet (129) through which biofluid that has passed through the device is recycled, if necessary. In some embodiments, the recycle inlet and the inlet are the same, in other embodiments they are separate inlets.

The inlet optionally contains a suitable connector to attach to a vessel, e.g. a bag or other container, a syringe, or the like, containing the nanoparticles, suspending agent, buffer, and/or other fluid to be fed into the reservoir. In those embodiments in which the recycle inlet is the same and the inlet, the connector also allows for the inlet to be in fluid communication with the biofluid to be recycled, such as by connecting to tubing that carries the biofluid. Optionally, the connector is a valve, such as a T-valve with two inlet ports and one outlet port.

b. Magnetic Filter

Various embodiments for the magnetic filter (10) are described above. In the preferred embodiment for the extracorporeal device, the magnetic filter contains an external magnet (12), preferably an electromagnet, a filter (14) formed from a magnetically attractive material, and a vessel (16).

In some embodiments, the device contains one magnetic filter. In other embodiments, the device contains more than one magnetic filter.

The screen is placed inside the vessel (16) and is, preferably, removable therefrom.

The vessel contains an inlet (17) and an outlet (18), where the inlet allows for the mixture of the biofluid and nanoparticles to enter the vessel and the outlet allows the filtrate to exit the vessel. In one embodiment, such as illustrated in FIG. 1A, the inlet (17) is connected to tubing (160), which is connects with a vessel that contains the mixture of the biofluid and the nanoparticles. In one embodiment, the outlet (18) is connected to tubing which connects with the pump (130).

In another embodiment, such as illustrated in FIG. 1B, the inlet (17) is connected to tubing (160), which, is attached via a valve and tubing to the pump (130). In one embodiment, the outlet (18) is connected to tubing which connects with a valve (180b) that either directs the flow of the biofluid to the reservoir for recycling or to the biofluid outlet (174) for delivery to the patient.

c. Pump

The pump (130) is designed to move the biological fluid through the various elements of the device and return it into the patient's body, following treatment. In one embodiment, the pump is in fluid communication, typically via tubing, with at least the magnetic filter and the reservoir. In another embodiment, the pump is in fluid communications with the mixing chamber, the reservoir and the magnetic filter.

Any pump which provides a suitable force for the desired flow rate may be used.

d. Mixing Chamber

In one embodiment, the biofluid is mixed with the nanoparticles in a mixing chamber (140) prior to entering the magnetic filter.

The mixing chamber contains one or more inlets (142) and typically one outlet (144). The mixing chamber is in fluid communication with the reservoir (120) and the biofluid inlet (172) through the one or more inlets (142).

In one embodiment, such as illustrated in FIG. 1A, the mixing chamber (140) is in fluid communication with the magnetic filter (10) through the outlet (144).

In another embodiment, such as illustrated in FIG. 1B, the mixing chamber (140) is in fluid communication with the pump (130) through the outlet (144).

While the nanoparticles and biofluid may enter the mixing chamber through two different tubes (160a and 160b), in one embodiment, they enter the mixing chamber (140) through one tube (160c). In this embodiment, the tubing (160a) connected to the biofluid inlet (172) connects with a first valve (180a) and the tubing connecting to the reservoir connects with the same first valve (180a). The first valve (180a) also contains an outlet (182a), which connects to tubing (160c) at the proximal end (162) of the tubing, where the distal end (164) of the tubing connects to the inlet to the mixing chamber (142).

e. Management Component

Preferably the device contains one or more management components, which include a computer with computer hardware and software for controlling various parameters for the device, including, but not limited to, the flowrate, fluid temperature, pressure, operating times, cycle times, and turning the electromagnet on or off.

The management component also typically contains a user interface for monitoring and manipulating the various parameters.

f. Housing

The housing (170) encases various components of the extracorporeal device. Preferably the housing contains a biofluid inlet (172) and a biofluid outlet (174), which are connectable to a patient, such as via tubing or other suitable connectors.

The housing may also contain a user interface for monitoring and manipulating the various parameters controlled by the management component. These components are in electrical communication or wireless communication with a processor, preferably in the management component.

g. Valves

Although not required, typically the device contains one or more valves to facilitate and control the flow of fluids through the various elements of the device.

As shown in FIG. 1A, preferably the device contains a first valve, such as a T-valve (180a), that connect the tubing (160a) connected to the biofluid inlet, the tubing (160b) connected to the reservoir and the tubing (160c) connected to the inlet to the mixing chamber.

Preferably, as shown in FIG. 1A, the device contains a second valve, such as a T-valve (180b), that connects the tubing (160f) connected to the outlet of the pump, which carries the filtrate from the magnetic filter, the tubing (160g) connected to the reservoir, which carries the recycled filtrate to the reservoir, and the tubing that connects to the biofluid outlet (174), which carries the filtrated biofluid and returns it to the patient via a suitable connector to the patient, e.g. a catheter or needle.

In another embodiment, the device may contain three or more valves. An exemplary configuration is illustrated in FIG. 1B, in which the device contains the first two valves described above, and contains a third valve (180c) that connects the tubing (160i) connected to the outlet of the pump, which carries the mixture of the biofluid and the nanoparticles after it exits the mixing chamber (140), the tubing (160k) connected to the reservoir, which carries the recycled mixture of the biofluid and the nanoparticles, and the tubing (160i) that connects to the magnetic filter (10), which carries the carries the mixture of the biofluid and the nanoparticles.

h. Tubing

The various fluids that are transported through the elements of the device, are typically contained within tubing (160) between each element. Any medical-grade tubing with a suitable diameter for the desired flowrate may be used. Suitable tubing materials include thermoplastics, such as polyvinylchloride, polycarbonate, polyurethane, and urethane, and tubing formed of mixtures or combinations thereof.

Suitable diameters range from outside diameters as large as a 0.350″ and inside diameters of 0.005″ or larger.

i. Optional Components

i. Magnetic Shielding

Optionally the magnetic filter is surrounded with a magnetic shield (200) to protect the patient and medical practitioners from the magnetic field. The magnetic shield draws the magnetic field into itself, providing a path for the magnetic field lines around the shielded volume. The effectiveness of this type of shielding decreases with the material's permeability, which generally drops off at both very low magnetic field strengths, and also at high field strengths where the material becomes saturated. To achieve low residual fields, the magnetic shield (200) may contain several enclosures one inside the other, each of which successively reduces the field inside it.

The shield has a suitable geometry to surround the magnetic filter. The preferred shape for the magnetic shields is a closed container that surrounds the magnetic filter. Any suitable shielding material may be used. Exemplary materials include Mu-metal™, MuShield™ high permeability magnetic shielding material (a non-oriented 80% nickel-iron-molybdenum alloy), GIRON™ Magnetic Shielding Film, and similar materials.

ii. Heater

The device may be temperature controlled. Optionally, the device contains a heater (210) to maintain the fluid flowing throughout the device and/or device at a selected temperature or temperature range.

iii. Sensors

Optionally, the device includes one or more pressure sensors for sensing the fluid pressure of the biofluid as it passes through the flow circuit and valves which regulate the flow of biofluid through the circuit, particularly where the purged biofluid is reintroduced into the subject's body.

In one embodiment, the device includes sensors, such as a photosensor or a similar detector to determine the presence of one or more magnetic, paramagnetic or superparamagnetic nanoparticles, and thereby direct the flow of the biofluid through the valve (180b) either to the biofluid outlet (174) or to the reservoir (120) to be recycled. If the sensor determines that one or more magnetic, paramagnetic or superparamagnetic nanoparticles are present in the biofluid, the management component will direct the flow through the valve to the reservoir (120) to be recycled. If the sensor determines that the biofluid is free of magnetic, paramagnetic or superparamagnetic nanoparticles, the management component will direct the flow through the valve to the biofluid outlet (174) to be returned to the patient. Thus, this sensor can prevent the superparamagnetic nanoparticles from entering the subject's circulatory system.

In one embodiment a sensor (220) for determining the presence of one or more magnetic, paramagnetic or superparamagnetic nanoparticles is located in the tubing that connects to the outlet of the pump (130) (see e.g. FIG. 1A). In another embodiment, the sensor (220) for determining the presence of one or more magnetic, paramagnetic or superparamagnetic nanoparticles is located in the tubing that connects to the outlet of the magnetic filter (10).

In one embodiment a sensor (230) for determining whether a sufficient level of complexation between the nanoparticles and the target cells or pathogen has occurred is located in the tubing that connects to the outlet of the pump (130) (see e.g. FIG. 1A). This data obtained by this sensor is typically communicated to the management component to direct the flow of the biofluid through the valve (180c) either to the magnetic filter (10) or to the reservoir (120) to increase the mixing time and allow for greater complexation prior to filtration.

iv. Siphon Tube or Sampling Port

Optionally, the device includes a siphon tube or sampling port in one or more flow lines to permit the removal of a biofluid sample for testing. In one embodiment, the device contains

B. Device for In Vivo Treatment

The extracorporeal device depicted in FIGS. 1A and 1B, can also be used as an in vivo device. However, prior to using the device, the nanoparticles are administered to the patient, typically parenterally, such as via an infusion, to the patient.

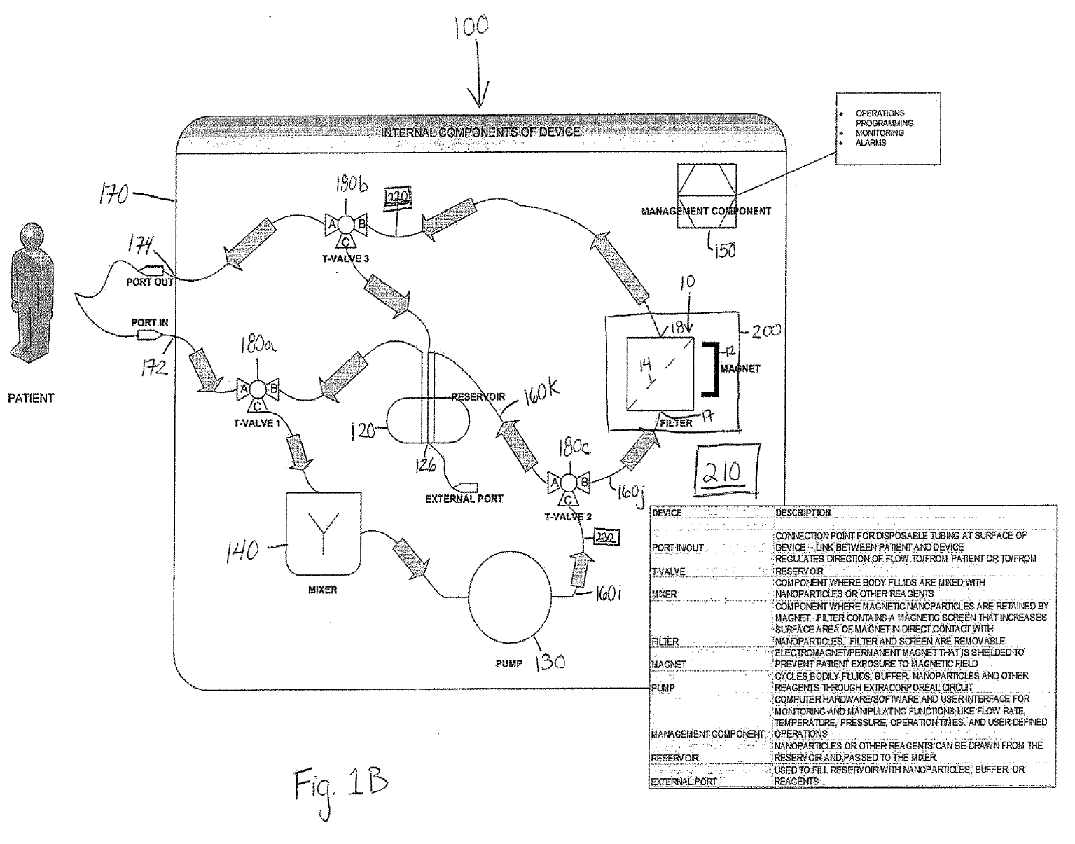

Alternative embodiments for the in vivo device are depicted in FIGS. 2A-2C. FIGS. 2A-2C illustrate modifications of standard hemodialysis or hemofiltration devices by introducing a magnetic filter (10) into the circuitry of the dialysis mechanism to remove magnetic nanoparticles from the fluids being extracted.

As shown in FIG. 2A, the patient's biofluid exists the patient's body and passes through a magnetic filter (10) through which the magnetic nanoparticles are removed. As shown in FIG. 2A, the magnetic filter (10) can be located in direct contact with the biofluid flow and adjacent to the location of the standard semi permeable membrane (220).

The magnetic filter can be placed anywhere in the circuit. As shown in FIG. 2B, the magnetic filter may be located at the beginning of the circuit prior to any other filters. As shown in FIG. 2C, the magnetic filter (10) may be included in a bypass to the circuit (240). In one embodiment, the by pass circuit may be included in the device to regulate the biofluid flow.

C. Combine Device with another Treatment System

Optionally, any one of the magnetic filter, extracorporeal device, or in viva device may be combined with another treatment system to provide a combination treatment.

### III. Systems

In one embodiment, a system containing a plurality of functionalized superparamagnetic nanoparticles and an extracorporeal device, in viva device, and/or magnetic filter, as described above, is provided.

A. Superparamagnetic Nanoparticles

Suitable superparamagnetic nanoparticles provide a large surface area for coupling to a selected ligand and have low non-specific absorption of biological materials. Additionally the nanoparticles do not interfere with the biological activity of the ligand. The nanoparticles also disperse well in the biological fluid into which they are introduced and only aggregate in the presence of an applied magnetic field.

The superparamagnetic nanoparticles may be coated with one or more biocompatible materials so as not to elicit an immunological response when introduced in viva.

The nanoparticles are typically functionalized with special components such as peptides, pseudo peptides, antibodies or other ligands that specifically bind to unattached malignant cells or blood-borne cancers. The functionalized particles could be introduced in a variety of manners, most notably intraperitoneally (to bind to residual malignant cells following abdominal surgery for cancer), or intravenously (to bind to blood-borne pathogens or leukemic cells). Physical extraction using the devices described above could be used to remove the particle/pathogen or particle/infected cell conjugates from the patient, leading to a substantially improved prognosis.

In other embodiments, the superparamagnetic nanoparticles are administered to a patient's biological fluid ex vivo. Preferably the superparamagnetic nanoparticles are administered in a suspension, which contains a suitable carrier. The carrier is a fluid which is physiologically compatible with the subject undergoing treatment, such as an isotonic phosphate-buffered saline (PBS) solution. Optionally, the carrier also contains heparin to prevent coagulation of the blood in the system. In another embodiment, the carrier also contains an effective amount of an antibiotic, such as penicillin or ampicillin, to reduce any bacterial growth which may be associated with the nanoparticles. The carrier is also preferably formulated such that it is at physiological pH.

In some instances, it may be necessary to agitate the nanoparticle suspension as the nanoparticles and liquid carrier are introduced in the flow circuit to ensure that the nanoparticles are relatively uniformly dispersed in the carrier.

Materials

a. Superparamagnetic

The nanoparticles are superparamagnetic. Superparamagnetic nanoparticles become magnetized in the presence of a magnetic field and remain demagnetized when it is withdrawn. Thus the particles do not aggregate until a magnetic field is applied. Superparamagnetic nanoparticles are particularly suited for use in the devices and methods described herein since they preserve the surface to volume ratio advantage when the particles are disperse in high numbers and, for in vivo applications prevent aggregation, which could lead to adverse physiological effects, such as embolism.

A superparamagnetic material is composed of small ferromagnetic clusters (e.g. crystallites), where the clusters are so small that they can randomly flip direction under thermal fluctuations. As a result, the material as a whole is not magnetized except in an externally applied magnetic field.

Superparamagnetism is a phenomenon in which magnetic materials may exhibit a behavior similar to paramagnetism at temperatures below the Curie or the Néel temperature. This is a small length-scale phenomenon, where the energy required to change the direction of the magnetic moment of a particle is comparable to the ambient thermal energy. At this point, the rate at which the particles will randomly reverse direction becomes significant.

Superparamagnetism can be verified using a hysteresis loop. Hysteresis loops typically compare the induced magnetization (M) to the strength of the applied external magnetic field (H). The magnetic field density B: can supplant the induced magnetization value (M) as seen in the figure. Remanence and coercivity can be determined from the hysteresis loop. Remanence is the magnetization that remains in the material once the magnetic field is removed and coercivity is the magnetic field strength needed to be applied in the opposite direction to return the material's magnetization to zero. Hysteresis is the work done on a magnet and is defined as the width of the hysteresis loop. The hysteresis measurements may be taken using a superconducting quantum interference device (SQUID) which is able to detect changes in magnetic field. Gallop, J. C., SQUIDs, the Josephson Effects and Superconducting Electronics. Adam Hilger: 1990.

For cell capture, using nanoparticles with stronger magnetic properties than Fe₃O₄ is generally required. Cobalt spinel ferrite, CoFe₂O₄ nanoparticles belong to the same spinel ferrite materials family as magnetite. They are preferred in one embodiment since they display much stronger magnetic responses at ambient temperatures than Fe₃O₄. (see Liu, C.; et al., “Chemical Control of Superparamagnetic Properties of Magnesium and Cobalt Spinel Ferrite Nanoparticles through Atomic Level Magnetic Couplings”, JACS, 122 (26) (2000); Song, Q.; Zhang, Z. J., Journal of Physical Chemistry B, 110 (2006)).

Other exemplary superparamagnetic spinel ferrites for forming the superparamagnetic particles include, but are not limited to: Cu₁₋ₓZnₓFe_{2-y}Ga_{y}O₄ with 0.0<x<0.5, 0<y<2, CuFe₂O₄, NiFe₂O₄, CoFe₂O₄, MgFe₂O₄, and Ni_{0.5}Cu_{0.5}Fe₂O₄ and Maganese oxide (Mn₃O₄).

Particle sizes required for the expression of superparamagnetic properties varies based on the material. See e.g. Sato et al., J. Magn. Magn. Mater., 65, 252 (1987). Typically, superparamagnetism is exhibited in particles with sizes of up to 100 nm. For example, the superparamagnetic nanoparticles may be MgFe₂O₄ spinel ferrite nanoparticles with the particle size of up to 50 nm.

Size of Functionalized Superparamagnetic Nanoparticles

The functionalized nanoparticles are typically less than 1 micron in size. Typically the functionalized nanoparticles range from about 20 nm until about and 1 micron depending on the coating used.

However, the functionalized superparamagnetic nanoparticles may be larger than 1 micron in some embodiments.

Shapes

The shape of the nanoparticles is selected to maximize the attachment of the binding partners to the surface of the nanoparticle. Thus, in a preferred embodiment, the nanoparticles are in the shape of smooth spheres.

At least one binding partner is attached to a functionalized nanoparticles. However, in one embodiment the binding partner coating on the nanoparticles preferably covers substantially all of the functionalized surface of the nanoparticle.

The particles may be spherical or non-spherical. In one preferred embodiment, the particles are spherical. In other embodiments, the particles may be non-spherical. For example, the nanoparticles may be oblong or elongated, nanotubes, nanorods, or have other shapes such as those disclosed in U.S. Publication No. 2008/0112886 and WO 2008/031035, entitled “Engineering Shape of Polymeric Micro- and Nanoparticles,” by S. Mitragotri, et al. and/or U.S. Publication No. 2006/0201390, entitled “Multi-phasic Nanoparticles,” by J. Lahann, et al.

The average diameter of a non-spherical particle is the diameter of a perfect sphere having the same volume as the non-spherical particle. If the particle is non-spherical, the particle may have a shape of, for instance, an ellipsoid, a cube, a fiber, a tube, a rod, or an irregular shape. In some cases, the particles may be hollow or porous.

Other shapes are also possible, for instance, core/shell structures (e.g., having different compositions), rectangular disks, high aspect ratio rectangular disks, high aspect ratio rods, worms, oblate ellipses, prolate ellipses, elliptical disks, UFOs, circular disks, barrels, bullets, pills, pulleys, biconvex lenses, ribbons, ravioli, flat pills, bicones, diamond disks, emarginate disks, elongated hexagonal disks, tacos, wrinkled prolate ellipsoids, wrinkled oblate ellipsoids, porous ellipsoid disks.

Biocompatible Coatings

The nanoparticles can be coated with a polysaccharide polymer or monosaccharide to increase their biocompatibility. This technique provides the advantage of diminishing an immune response to the particles since glycans do not typically illicit such a response (Lacava, et al., Journal of Magnetism and Magnetic Materials, 272-276, 2434-2435 (2004)). The polymer coating also contains numerous free hydroxyls that willingly form hydrogen bonds in aqueous solution. In concert, the many surface hydroxyls hold the particle and surface coat in suspension for an indefinite period of time. The coating is preferred in those embodiments in which the nanoparticles are injected into the general circulation or the ascities fluid of the peritoneal cavity.

Suitable coating materials include, but not are not limited to, silanes, such as polydimethylsiloxane, silicon oil, silicones, vinylsilane graft copolymers, in which a biocompatible material is grafted to the vinyl silane, such as those listed above; saccharides, polysaccharides, and derivatives thereof, such as dextran, glucuronic acid, polygalacturonic acid, chitosan, neuraminic acid, agar, agarose, alginates, carrageenan, celluloses and modified celluloses, condroitin, hyaluronic acid, pectin, starch, xanthan, and combination thereof. Alternative coating materials include, but are not limited, to non-degradable, biocompatible polymers, such as poly(alkylene oxides), such as PEG, PPO, and copolymers thereof, polyurethanes, biocompatible acrylates and alkylacrylates, such as methacrylates and hydroyalkyl methacrylates, polyalkylenes, such as polyethylene, polypropylene, and polytetrafluoroethylene, polyvinyl alcohols, polyvinylacetates, poly(ethylene-co-vinylacetate), polyesters, such as poly(ethylene terephthalate), poly(sulfones). Alternative coating materials include, but are not limited, biodegradable, biocompatible polymers, such as PLA, PGA, and copolymers thereof, poly(p-dioxanone) and copolymers thereof, polycaprolactone, polyhydroxyalkanoates, polyanhydrides, poly(orthoesters), polyphosphazines, poly(alkylcyanoacrylates), and proteins, such as gelatin. Further, the coating may contain Surfactants, such as Tweens, poloxamers, pluronics, tetronics.

Functionalization

The nanoparticles can be functionalized with one or more binding partners for targeting cells expressing or displaying corresponding surface membrane binding partners, for example cancer cells or to specific pathogenenic agents or cells infected by a pathogenic agent.

As used herein, the term “binding partner” refers to a molecule that can undergo binding with another particular molecule. For example, the binding may be highly specific and/or non-covalent. Binding partners which form highly specific, non-covalent, physiochemical interactions with one another are defined herein as “complementary binding partners”. Non-limiting examples include nucleic acid-nucleic acid binding, nucleic acid-protein binding, protein-protein binding, enzyme-substrate binding, receptor-ligand binding, receptor-hormone binding, antibody-antigen binding, etc.

As additional examples, binding partners may include antibody/antigen pairs, ligand/receptor pairs, enzyme/substrate pairs and complementary nucleic acids or aptamers. Suitable antibodies for use as binding partners include antigen-binding fragments, including separate heavy chains, light chains Fab, Fab′, F(ab′)₂, Fabe, and Fv. Antibodies also include bispecific or bifunctional antibodies.

Binding partners can be attached covalently or through non-covalently bonding strategies, such as but not limited to dichalcogenide bonding.

As used herein, “the first binding partner” generally refers to a binding partner attached to or associated with a superparamagnetic nanoparticle.

As used herein “functionalized superparamagnetic nanoparticle” refers to a superparamagnetic nanoparticle that contains a plurality of binding partners attached to or associated with the surface of the nanoparticle.

As used herein, the term “pathogenic agent” is defined broadly and includes, without limitation, viruses, virally infected cells, bacteria, and other particles or organisms the presence of which is toxic or otherwise undesirable in a biological host. One way to target the nanoparticle to a cancer cell or pathogenic agent or cell infected with a pathogenic agent is via the attachment of a binding partner that is specific for antigens preferentially expressed by the cells, to the nanoparticle surface.

The antigen expressed by the tumor may be specific to the tumor, or may be expressed at a higher level on the tumor cells as compared to non-tumor cells. The association of the binding partner to the nanoparticle can be by a covalent or ionic bond or other non-covalent interaction. U.S. Pat. No. 5,601,800 describes several methods for attaching biologically active agents, such as diagnostic agents, contrast agents, receptor agents, and radionuclides to particles.

Useful linkers and methods of use are described in, for example, U.S. Pat. No. 5,824,805; U.S. Pat. No. 5,817,742; and U.S. Pat. No. 6,339,060. The nanoparticles may be functionalized via direct covalent attachment of binding partners to functional groups on the nanoparticle coating. The binding partners may also be covalently attached, conjugated or linked to the nanoparticle via a linker. The linker may be synthetic or natural, and can comprise a short peptide or a small polymer, such as a triethylene glycol polymer or a polyethylene glycol polymer.

a. Binding Partners Attached to the Nanoparticles

The binding partner attached to the nanoparticles may be an antibody, or fragment thereof, recognizing a specific surface antigen. Such immunoconjugates allow the selective delivery of the functionalized superparamagnetic nanoparticle to targeted cells expressing or displaying the antigen. (See, e.g., Hermentin and Seiler, Behringer Insti. Mitl., 82:197-215 (1988); Gallego et al., Int. J. Cancer 33:773744 (1984); Amon et al., Immunological Rev. 62:5-27 (1982)). Antigens can be peptides, proteins, polysaccharides, saccharides, lipids, nucleic acids, or combinations thereof. The antigen can be derived from a virus, bacterium, parasite, plant, protozoan, fungus, tissue or transformed cell such as a cancer or leukemic cell and can be a whole cell or immunogenic component thereof, e.g., cell wall components or molecular components thereof.

Suitable antigens are known in the art and are available from commercial government and scientific sources. In one embodiment, the antigens are whole inactivated or attenuated organisms. These organisms may be infectious organisms, such as viruses, parasites and bacteria. These organisms may also be tumor cells. The antigens may be purified or partially purified polypeptides derived from tumors or viral or bacterial sources. The antigens can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids.

For example, the binding partner attached to the nanoparticle may be an antibody or antibody fragment recognizing a tumor antigen. The antibody-containing nanoparticle can then be located at a tumor site by both a magnetic field and by antibody-ligand interactions.

Antibodies and antibody fragments, including monoclonal antibodies, anti-idiotype antibodies, and Fab, Fab′, F(ab′)₂ fragments or any other antibody fragments, that recognize a selected antigen can be obtained by screening antibodies and selecting those with high affinity. (See, generally, U.S. Pat. Nos. RE 32,011; 4,902,614; 4,543,439 and 4,411,993; see also, Monoclonal Antibodies, Hybridonzas: A New Dimension in Biological Analyses, Plenum Press, Kennett, McKeam, and Bechtol (eds.), 1980; Antibodies: A Laboratory Manual, Harlow and Lane (eds.), Cold Spring Harbor Laboratory Press, 1988)).

Alternatively, antibodies or antibody fragments may also be produced and selected utilizing recombinant techniques. (See, e.g., Huse et al., Science 246:1275-1281 (1989); Sastry et al., Proc. Natl. Acad. Sci. USA, 86:5728-5732 (1989); Alting-Mees et al, Strategies in Molecular Biology, 3:1-9 (1990)). In addition, antibodies that selectively bind to an antigenic determinant of a surface protein on known pathogens (e.g., gp 120, on the HIV virus coat), are commercially available or can be prepared using conventional techniques.

The binding partner attached to the nanoparticles can be a ligand recognized by cell-specific receptors. For example, neuraminic acid or sialyl Lewis X can be attached to a superparamagnetic nanoparticle. Such a ligand-containing nanoparticle can then be located at a specific site, such as an endothelial site, by both a magnetic field and by ligand-selection interactions. Such conjugates are suitable for the treatment or prophylaxis of diseases in which bacterial or viral infections, inflammatory processes or metastasizing tumors are involved.

Other ligands, such as protein or synthetic molecules that are recognized by receptors can be associated with the superparamagnetic nanoparticles. In addition the binding partner attached to or associated with the superparamagnetic nanoparticles may be a peptide, DNA and/or RNA recognition sequence.

The term “aptamers” as used herein refers to nucleic acids (typically DNA, RNA or oligonucleotides) or peptides that bind to a specific target molecule. Methods for making and modifying aptamers, and assaying the binding of an aptamer to a target molecule are known to those of skill in the art (see for example, U.S. Pat. Nos. 6,111,095, 5,861,501, 5,840,867, 5,792,613, 5,780,610, 5,780,449, 5,756,291 5,631,146 and 5,582,981; as well as PCT Publication Nos. WO92/14843, WO91/19813, and WO92/05285, each of which is incorporated herein by reference). Ligands that bind aptamers include but are not limited to small molecules, peptides, proteins, carbohydrates, hormones, sugar, metabolic byproducts and toxins. Aptamers configured to bind to specific target can be selected, for example, by synthesizing an initial heterogeneous population of oligonucleotides, and then selecting oligonucleotides within the population that bind tightly to a particular target molecule. Once an aptamer that binds to a particular target molecule has been identified, it can be replicated using a variety of techniques known in biological and other arts, for example, by cloning and polymerase chain reaction (PCR) amplification followed by transcription.

b. Binding Partners on the Surface Membrane of a Target Cell

The target cell may contain one or more binding partners on its surface. Binding partners that may be on the surface of the cells include, but are not limited to, cancer antigens, viral antigens, bacterial antigens, protozoan antigens, and fungal antigens.

i. Cancer Antigens

The disclosed nanoparticles can be functionalized with binding partners that bind to proteins on the surface of cancer cells and are not on the surface of non-cancer cells. Exemplary cancer specific proteins include, but are not limited to cancer antigens also referred to as tumor specific antigens. For example, the receptor tyrosine kinase, EphA2, is expressed prolifically in patients with ovarian carcinoma. The peptide sequence YSAYPDSVPMMS (SEQ ID NO:1) acts as an ephrin mimetic and is highly selective for EphA2. Nanoparticles functionalized with a derivative of this peptide sequence (GGGGYSAYPDSVPMMSK) (SEQ ID NO:2) preferentially bind cells in populations testing positive for EphA2 expression.

Additional cancer antigens include human epithelial antigen (HEA) and the extracellular domain of the MUC16 cell surface protein, CA125, which have been investigated extensively as potential diagnostic aids for identifying malignant adenocarcinomas in serous effusions. Human epithelial antigen (HEA) is a glycoprotein epitope that exhibits elevated expression levels in metastatic adenocarcinomas. Ber-EP4 has a high affinity for HEA and is commonly used to verify HEA expression. Nanoparticles functionalized with Ber-EP4 cells should preferentially bind cells expressing CA125.

Serum levels of CA125 are elevated in 90% of the patients with ovarian cancer. HEA and CA125 levels were expected to be elevated in the same cell populations expressing high levels of EphA2.

Although increased EphA2 expression has been linked to ovarian carcinomas, it is not exclusively expressed by this cell type. The Eph receptor family is one of the largest RTK families and Eph/ephrin receptor signaling is intimately coupled with cell movement, maintenance of cellular boundaries, and blood vessel remodeling. An ephrin mimetic is expected to bind any cell expressing an Eph receptor but is also expected to preferentially bind cells with higher Eph expression.

Other antigens specific for cancer cells can similarly be targeted using ligand that specifically bind to these antigens. For example, nanoparticles bearing ligands or antibodies to the transferrin receptor which is overexpressed on many cancer cell would target the nanoparticles to those cells. Similarly, nanoparticles bearing antibodies against MUC1, MUC1 an ErbB receptor or any other growth factor receptor would aid in targeting the nanoparticles to cancer cells. Cell surface proteins that also make desirable targets for agents attached to nanoparticles laden with therapeutic agents include PSA, TACE, MMP-14, CEA (carcinoembryonic antigen widely overexpressed in a wide variety of cells), Urokinase receptor (overexpression is strongly correlated with poor prognosis in a variety of malignant tumors) and CXCR4 (linked to breast cancer invasion and metastasis). Other proteins that make desirable targets for targeted delivery of therapeutic agents include immune system markers such as CD3, CD2, Fc gamma R activating receptor (CD16), some superantigens, glycosyltransferase-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession No. X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor-associated antigens that can be targeted may also include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). The tumor associated antigen, mesothelin, defined by reactivity with monoclonal antibody K-1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)).

Tumor antigens of known structure and having a known or described function include the following cell surface receptors: HER1 (GenBank Accession No. U48722), HER2 (Yoshino, et al., J. Immunol., 152:2393 (1994); Disis, et al., Cane. Res., 54:16 (1994); GenBank Acc. Nos. X03363 and M17730), HER3 (GenBank Acc. Nos. U29339 and M34309), HER4 (Plowman, et al., Nature, 366:473 (1993); GenBank Acc. Nos. L07868 and T64105), epidermal growth factor receptor (EGFR) (GenBank Acc. Nos. U48722, and KO3193), vascular endothelial cell growth factor (GenBank No. M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Acc. Nos. M38651, X03635, X99101, U47678 and M12674), progesterone receptor (GenBank Ace, Nos. X51730, X69068 and M15716), follicle stimulating hormone receptor (FSH-R) (GenBank Acc. Nos. 234260 and M65085), retinoic acid receptor (GenBank Acc. Nos. L12060, M60909, X77664, X57280, X07282 and X06538), MUC-1 (Barnes, et al., Proc. Nat. Acad. Sci. USA, 86:7159 (1989); GenBank Acc. Nos. M65132 and M64928) NY-ESO-1 (GenBank Acc. Nos. AJ003149 and U87459), NA 17-A (PCT Publication No. WO 96/40039), Melan-A/MART-1 (Kawakami, et al., Proc. Nat. Acad. Sci. USA, 91:3515 (1994); GenBank Acc. Nos. U06654 and U06452), tyrosinase (Topalian, et al., Proc. Nat. Acad. Sci. USA, 91:9461 (1994); GenBank Acc. No. M26729; Weber, et al., J. Clin. Invest, 102:1258 (1998)), Gp-100 (Kawakami, et al., Proc. Nat. Acad. Sci. USA, 91:3515 (1994); GenBank Acc. No. 573003, Adema, et al., J. Biol. Chem., 269:20126 (1994)), MAGE (van den Bruggen, et al., Science, 254:1643 (1991)); GenBank Acc. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Acc. No. U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175, U90842, U90841 and X86174), carcinoembryonic antigen (CEA, Gold and Freedman, J. Exp. Med., 121:439 (1985); GenBank Acc. Nos. M59710, M59255 and M29540), and PyLT (GenBank Acc. Nos. 302289 and J02038); p97 (melanotransferrin) (Brown, et al., J. Immunol.,127:539-46 (1981); Rose, et al., Proc. Natl. Acad. Sci. USA, 83:1261-61 (1986)). Other tumor-associated and tumor-specific antigens are known to those of skill in the art and are suitable for targeting using the disclosed nanoparticles.

ii. Viral Antigens

A viral antigen can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxyiridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens may be derived from a particular strain such as a papilloma virus, a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

Bacterial Antigens

Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chrornatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

iv. Antigens of Parasites

Antigens of parasites can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

Therapeutic or Diagnostic Agent

In some embodiments, one or more therapeutic agents are incorporated with the superparamagnetic nanoparticles for delivery to specific sites under control of a magnetic field. A therapeutic agent can be incorporated with the superparamagnetic nanoparticles by a linkage. For example, a therapeutic agent can be covalently linked to the polymer, either directly or through a linker. Alternatively, a therapeutic agent can be ionically linked, or associated, to the polymer, either directly or through a linker or a derivative.

Therapeutic agents include, but are not limited to, small molecules, macromolecules, peptides, proteins, enzymes, DNA, RNA, genes, cells, or radionuclides. The therapeutic agents have one or more therapeutic properties when administered to a patient in an effective amount. Non-limiting examples of therapeutic properties are antimetabolite, antifungal, anti-inflammatory, antitumoral, antiinfectious, or antibiotic. A therapeutically effective amount of a therapeutic agent associated with the nanoparticles will be determined by one skilled in the art as that amount necessary to effect treatment of a particular disease or condition, taking into account a variety of factors such as the patient's weight, age, and general health, the therapeutic properties of the drug, and the nature and severity of the disease

Targeting the therapeutic agent to tumor cells may be accomplished by linking the therapeutic agent to the nanoparticles via a peptide sequence that is cleavable by an enzyme associated with a cancer. For example, expression of matrix metalloproteases such as matrix metalloprotease 9 (MMP-9), matrix metalloprotease 13 (MMP-13) and matrix metalloprotease (MMP-2) have been found to be elevated in a number of human epithelial cancers, including breast (Davies et al., British Journal of Cancer 67:1126, 1993); prostate (Harndy et al., British Journal of Cancer 69:177, 1994); colon (Levy et al., Cancer Research 51:439, 1991); ovary (Naylor et al., International Journal of Cancer 58:50, 1994); bladder (Davies et al., British Journal of Cancer 67:1126, 1993); and gastric carcinoma (d'Errico et al., Mod Pathol. 4:239, 1991). The optimal MMP-2 cleavage motif such as IPVGLIG (SEQ ID NO:3), and IVSLRS (SEQ ID NO:4) are disclosed in Turk et al., Nature Biotechnology 19:661, (2001). Thus, functionalizing the therapeutic agent to the nanoparticle with a linker comprising IPVGLIG (SEQ ID NO:3) or IVSLRS (SEQ ID NO:4) should result in release of the agent from the nanoparticle by MMP-2. The linker may comprise a PQGLA (SEQ ID NO:5) sequence that is cleavable by metalloproteinase-13 enzyme (Kim, et al., Biomacromolecules 4(4):1214-1223 (2003). Additional peptides cleavable by metalloproteases are disclosed in WO 01/68145. Other examples include, linkages targeted to other cancer-cell specific or overexpressed proteases, for example, prostate-specific antigen (PSA), which have sequence-specific proteolytic capabilities.

a. Chemotherapeutic Agents

More specifically, therapeutic agents that can be attached to the nanoparticles include chemotherapeutic agents such adriamycin, dactinomycin, bleomycin, vinblastine, cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-Ia; interferon gamma-Ib; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole.

Other chemotherapeutic agents include antibodies such as HERCEPTIN® (Trastuzumab) (Genentech, Calif.) which is a humanized anti-HER2 monoclonal antibody for the treatment of patients with metastatic breast cancer; REOPRO® (abciximab) (Centocor) which is an anti-glycoprotein IIb/IIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Phaun/Genentech, Roche/Zettyaku); LYMPHOCIDE™ which is a humanized anti-CD22 IgG antibody (Immunomedics); LYMPHOCIDE™ Y-90 (Immunomedics); Lymphoscan (Tc-99m-labeled; radioimaging; Immunomedics); Nuvion (against CD3; Protein Design Labs); CM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-a Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Gemmab); CD20-sreptdavidin (+biotin-yttrium 90; NeoRx); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

The therapeutic agents for the targetable nanoparticles may also be radioisotopes. Such radioisotopes are chemical compounds or elements that emit alpha, beta or gamma radiation and that are useful for diagnostic and/or therapeutic purposes. One factor used in selecting an appropriate radioisotope is that the half-life be long enough so that it is still detectable or therapeutic at the time of maximum uptake by the target, but short enough so that deleterious radiation with respect to the host is minimized. Selection of an appropriate radioisotope would be readily apparent to one having ordinary skill in the art. Generally, alpha and beta radiation are considered useful for local therapy. Examples of useful therapeutic compounds include, but are not limited to ³²P, ¹⁸⁶Re, ¹⁸⁸Re, ¹²³I, ¹²⁵I, ¹³¹I, ⁹⁰Y, ¹⁶⁶Ho, ¹⁵³Sm, ¹⁴²Pr, ¹⁴³Pr, ¹⁴⁹Tb, ¹⁶¹Tb, ¹¹¹In, ⁷⁷Br, ²¹²Bi, ²¹³Bi, ²²³Ra, ²¹0Po, ¹⁹⁵Pt, ^{195m}Pt, ²⁵⁵Fm, ¹⁶⁵Dy, ¹⁰⁹Pd, ¹²¹Sn, ¹²⁷Te, ¹⁰³Pd, ¹⁷⁷Lu, and ²¹¹At. The radioisotope generally exists as a radical within a salt, although exceptions such as iodine and radium exist wherein the radical is not in ionic form.

a. Fluoresce, Preferably Following Binding to Cancer Cell, Or Other Pathogen.

For diagnostic or imaging purposes, the binding partner attached to the nanoparticle may additionally be functionalized to an agent such as a diagnostic isotope or a fluorophore, which can be visualized following binding to the cancer cells. In this embodiment, the fluorescent compound may be separated from the binding domain of the ligand by a short amino acid chain. For example a Rhodamine tag can be conjugated on the N-terminus of the ligand, and four N-terminal glycine residues used to distance the Rhodamine from the binding region and prevent steric hindrance of the antigenic agent binding. There are many rhodamine derivatives used for imaging purposes, for example tetramethylrhodamine (TAMRA) and its isothiocyanate derivative (TRITC) and, sulforhodamine 101 (and its sulfonyl chloride form Texas Red) and Rhodamine Red. TRITC is the base rhodamine molecule functionalized with an isothiocyanate group (—N═C═S), replacing a hydrogen atom on the bottom ring of the structure. This derivative is reactive towards amine groups on proteins inside cells. A succinimidyl-ester functional group attached to the rhodamine core, creating NHS-rhodamine, forms another common amine-reactive derivative. Other derivatives of rhodamine include newer fluorophores such as Alexa 546, Alexa 555, Alexa 633, DyLight 549 and DyLight 633, have been tailored for various chemical and biological applications where higher photostability, increased brightness, different spectral characteristics, or different attachment groups are needed.

Useful diagnostic radioisotopes exist, and are well-known to those ordinarily skilled in the art. The useful diagnostic and therapeutic radioisotopes may be used alone or in combination.

### III. Methods of Using Device and/or Magnetic Nanoparticles

The devices and nanoparticles described herein may be used to filter the blood, peritoneal fluids, lymph, cerebrospinal fluids, or other serous fluids in the body. In one embodiment, the devices and nanoparticles are used to remove cancer cells from the circulation or peritoneal cavity. Alternatively, the devices and functionalized superparamagnetic nanoparticles may be used to treat and remove blood-borne cancer metastases and leukemias.

In one embodiment, either the in vivo or the extracorporeal device may be used to extract cancer cells from a patient. Both the in vivo and extracorporeal devices are particularly suited for removal of metastatic cancer cells. The use of the devices in combination with the appropriately functionalized superparamagnetic nanoparticles may reduce the metastatic spread of cancer cells from primary carcinomas.

In one embodiment, the device may be combined with other treatment methods, such as surgery to remove a tumor and/or chemotherapy to shrink a tumor.

In another embodiment, following removal of the cancer cells from a biofluid by the magnetic filter, the cells are removed from the filter and analyzed and characterized by suitable methods. This step may be used to further determine if/which types of additional cancer treatments may be helpful to the patient.

Ovarian Cancer

Ovarian cancer is one of the most lethal gynecological malignancies. The survival rate for patients with late stage disease is about 20%. Because of the difficulty in early diagnosis of ovarian cancer, 81% of all cases are detected in late stages with metastatic spread of malignant cells. The most significant pathway of ovarian tumor spread occurs via exfoliation of malignant cells from primary tumor sites, leading to dissemination of cancer cells throughout the peritoneal cavity (Chi, and Hoskins, W. J., Ovarian Cancer Methods and Protocols. Humana Press: 2000; p 75) and worsening the prognoses for cancer patients (Hanahan & Weinberg, Cell, (1):57-70 (2000), Fidler, Nat. Rev. Cancer, 3(6):453-8 (2003). In addition, some cancer cells may escape during primary tumor excision, and the development of resistance in these cells to current chemotherapies can lead to regrowth of a tumor cell population. Intraoperative rupture of malignant epithelial ovarian neoplasms also has been shown to worsen the prognosis of patients with early stage ovarian cancer (de la Cuesta, et al., Obstet. Gynecol. 84(1):1-7 (1994). Thus, combining the extraction of residual tumor cells to limit the metastatic spread as part of routine treatment procedures could be a strategy to improve long-term survival for cancer patients.

In ovarian cancer cases, significant removal of disseminated cancer cells from the abdominal cavity could lead to reduction of the malignant cell population and reduce the odds of metastatic spread. Additional studies, including evaluation of toxic effects from magnetic nanoparticles, are needed before this method can advance to clinic trial stage. Further improvement of this concept may include refinement of the extraction process with an array of peptides using patient specific tumor protein expression profiles. Since small peptides have been reported to prevent tumor cell adherence onto tissues in a murine model using a bladder tumor cell line (Goldstein, et al, J Endourol, 7(3):237-41 (1993), they might also be incorporated into the magnetic cell extraction technique to reduce the possibility of tumor implants and therefore greatly enhance the efficiency of preventing metastatic spread of cancer. Since EphA2 is also highly expressed in other types of cancers, applications of the YSA peptide-magnetic nanoconjugates could be expanded beyond ovarian cancers.

Any cancer may be treated using the functionalized nanoparticles described herein. These include but are not limited to adrenocortical carcinoma, anal, bladder cancer, breast, cervical, colon, endometrial, esophageal, extrahepatic bile duct, eye, gallbladder, gastric, head and neck, hypopharyngeal cancer, renal, laryngeal, lip and oral cavity, liver, lung, naso- or oropharyngeal, oral, ovarian, pancreatic, parathyroid, penile, pheochromocytoma, pituitary, prostrate, rectal, skin, thyroid, vaginal or vulval cancers, brain tumor, carcinoid tumor, gastrointestinal carcinoma, Ewings Family of Tumors (PNET), extracranial germ cell tumor, childhood eye cancer, intraocular melanoma, germ cell tumor, extragonadal gestational trophoblastic tumor, islet cell carcinoma, leukemia, lymphoma, hodgkins and non-hodgkins disease, mesothelioma, melanoma, merkel cell carcinoma, mycosis fungoides, myelodysplastic Syndrome, myeloproliferative disorders, neuroblastoma, osteosarcoma, rhabdomyosarcoma, malignant thymoma, and Wilms' tumor.

Furthermore, this in vivo extraction approach utilizing superparamagnetic nanoparticles may also be used in principle for the treatment of viral diseases by targeting and removing viruses and virus-infected cells and therefore bolster the immune system to fight infections.

A. Ex Vivo Device

The following method is described with reference to the device schematic illustrated in FIG. 1A. Preferably the biofluid is removed from the subject via an inlet port, such as a needle or catheter, or the like, which is connected to tubing. The tubing attaches at its proximal end to the inlet port and at its distal end to the device. Typically this tubing is removable from the device to facilitate disposal after use by a subject.

In one embodiment, the method includes the perfusion of biofluid from a subject and the introduction of a plurality of the functionalized superparamagnetic nanoparticles into the biofluid.

Biofluid flow from the subject is preferably controlled by the first valve (180a). In this embodiment, the method and device provide continuous flow of biofluid from a subject, through a flow circuit, whereby purged biofluid is returned to the subject at substantially the same flow rate at which it leaves the subject. The functionalized superparamagnetic nanoparticles are pumped out of the reservoir (120) and mixed with the biofluid. Typically, as illustrated in FIG. 1A, the nanoparticles pass through the first valve (180a) and the biofluid and the nanoparticles exit the first valve and enter the mixing chamber (140) to ensure thorough mixing.

The quantity or number of functionalized superparamagnetic nanoparticles which are introduced into the biofluid will vary depending upon the level of metastatic cancer cells, or cells infected with a virus. Therefore, the level of metastatic cancer cells, or cells infected with a virus is preferably determined prior to the introduction of functionalized superparamagnetic nanoparticles into the biofluid. If necessary, additional functionalized superparamagnetic nanoparticles can be added to the reservoir to ensure that each cancer cell or virally infected cell is bound to at least one functionalized superparamagnetic nanoparticle.

To facilitate the introduction and metering of the plurality of functionalized superparamagnetic nanoparticles into the flow system, it is preferred that a suspension of the functionalized superparamagnetic nanoparticles be prepared and stored in the reservoir (120).

Next, biofluid and the functionalized superparamagnetic nanoparticles flow into mixing chamber (140) for mixing or agitating the biofluid with the functionalized superparamagnetic nanoparticles to promote contacting of the cancer cells by the ligand. The mixture of biofluid and functionalized superparamagnetic nanoparticles remains in the mixing chamber for a suitable period of time to ensure that the target cells contact and are bound to one or more functionalized superparamagnetic nanoparticles to form a complex. The probability of binding upon contact and the strength of the bond between the binding partners attached to the nanoparticles and the receptors on the surface of the cells are functions of the affinity of the binding partners for the receptor and the time in which they are in contact with each other. Preferably at least 50%, more preferably greater than 50% of the target cells and are bound to one or more functionalized superparamagnetic nanoparticles to form a complex to reduce the number of cycles necessary to completely filter the fluid. By way of example, studies showed that 100% of the cancer cells expressing EphA2 could be sequestered with the peptide function after 15 minutes in the extracorporeal device.

Next step, the biofluid, nanoparticle-cell complexes and any free nanoparticles and uncomplexed cells flow out of mixing chamber and into the inlet of the vessel for the magnetic filter.

The magnetic filter includes a vessel, with an inlet and an outlet. Preferably, the vessel also includes a screen. Preferably a magnet is external to the vessel. The magnet generates a magnetic field. In one embodiment, the magnet is one or more permanent magnets. In another embodiment, the magnet is an electromagnet. Preferably, the magnetic field magnetizes the screen to provide a greater surface area upon which the nanoparticles can attach.

When the biofluid with the nanoparticle-cell complexes contacts the screen, the free nanoparticles and/or nanoparticle-cell complexes adhere to the screen. In this fashion, the complexes are restrained against the continuous forward flow of biofluid through magnetic filter. Optionally, the flow may be stopped at a given point, and the filter may be removed from the device for removal of the free nanoparticles and the nanoparticle-cell complexes, and optionally testing of the nanoparticle-cell complexes.

If any free nanoparticles and/or nanoparticle-cell complexes do not adhere to the screen, then they may flow out of the magnetic filter with the filtrate, and optionally, may be introduced into the reservoir and recycled through the mixing chamber and magnetic filter one or more times to ensure that all nanoparticles have been removed prior to returning the filtrate to the patient. To confirm that no superparamagnetic nanoparticle leave the magnetic filter with the purged biofluid, a photosensor or a similar detector (220) may be provided. If the sensor (220) detects one or more superparamagnetic nanoparticles, the sensor will send a signal to the management component and the second valve (180b) will be closed with respect to the line that transmits biofluid to the patient so that the filtrate is sent to the reservoir (120) for recirculation. This prevents the superparamagnetic particle from entering the subject's circulatory system.

Optionally, the device contains more than one magnetic filter. Optionally the magnetic filters are configured in side-by-side relationship and a directional valve system alternates flow of biofluid containing the nanoparticle-cell complexes between the filters to allow magnetically adhered complexes to be removed from one magnetic filter while more complexes are collected in the second filter. In other words, biofluid can be intermittently channeled between the two or more magnetic filters to allow cleaning of the filters without disrupting biofluid flow through the circuit.

Finally, when the biofluid leaves the one or more magnetic filters, it is substantially purged of the preselected cancer cells. To verify that the biofluid has been adequately purged, the device may contain a second siphon tube or sampling port following magnetic filter.

Flow line (x) is provided which leads directly into the subject such that the entire system is a continuous flow circuit similar to that used in blood dialysis systems.

a. Dosing of Superparamagnetic Nanoparticles

Any suitable concentration and volume of functionalized superparamagnetic particles can be used in the device and method described herein. The amount and volume used are functions of the treatment required. By way of example, in one embodiment, the concentration of nanoparticles will be sufficient to provide 1-100 nanoparticles per target cell or pathogen.

b. Flow Rate

Flowrates are selected based on the treatment. They are designed to match accepted standards outlined for hemofiltration, hemodialysis, and peritoneal dialysis procedures and will vary depending on the type and physical properties of the fluid (i.e. viscocity) and the physical properties of the patient (i.e. age, weight, height, sex).

B. In Vivo Treatment

The in vivo treatment steps are similar to the ex vivo treatment steps, with the exception that the device typically does not contain a mixing chamber and therefore does not include the step of mixing for a suitable period of time to allow for complexation. Further, prior to removal of the biofluid from the patient, the functionalized superparamagnetic nanoparticles are first administered in the patient, typically via injection. After a suitable period of time has passed to allow for complexation of the cancer cells to the superparamagnetic particles, a portion of the biofluid is removed from the patient, at a suitable flow rate, and is administered to the in vivo device for removal of the superparamagnetic nanoparticles, and complexes thereof.

a. Dosing of Functionalized Superparamagnetic Nanoparticles

Any suitable concentration and volume of functionalized superparamagnetic particles can be used in the device and method described herein. The amount and volume used are functions of the treatment required. By way of example, in one embodiment, the concentration of nanoparticles will be sufficient to provide 1-100 nanoparticles per target cell or pathogen.

b. Flow Rate

Flowrates are selected based on the treatment. They are designed to match accepted standards outlined for hemofiltration, hemodialysis, and peritoneal dialysis procedures and will vary depending on the type and physical properties of the fluid (i.e. viscocity) and the physical properties of the patient (i.e. age, weight, height, sex).

C. Removal of Superparamagnetic Nanoparticles

In one embodiment, the in vivo device may be used to remove magnetic, paramagnetic or superparamagntic nanoparticles that have been administered to a patient. In another embodiment, the superparamagnetic nanoparticles may be removed from a patient, by adding a magnetic filter to an existing hemofiltration or dialysis device (see e.g. FIGS. 2A-2C).

In another embodiment, the extracorporeal device may be used to remove biological fluids from a patient to whom magnetic, paramagnetic or superparamagnetic nanoparticles nanoparticles were previously administered. In this embodiment, the reservoir may be used to add chemotherapeutic agents, and control fluid levels in body cavities like the peritoneum (i.e. draining and filling the cavity), if needed. If the reservoir is not required, the valve (180a) can be closed with respect to the reservoir. In this embodiment, the mixing chamber may also be optional.

In one embodiment, the magnetic, paramagnetic, or superparamagnetic nanoparticles are first administered to the patient to deliver a therapeutic or diagnostic agent. In one embodiment, the magnetic, paramagnetic, or superparamagnetic nanoparticles may contain a fluorescent dye and a suitable ligand along with a thrombolytic agent to breakup a blockage in a patient's artery. After delivery of an effective amount of the thrombolytic agent to the blockage, the clot dissolves, leaving magnetic, paramagnetic, or superparamagnetic nanoparticles in the patient's body. These particles can be removed using any of the devices described herein.

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1

### In Vitro Capture of Murine Cancer Cells with Superparamagnetic CoFe₂O₄ Nanoparticles Coated with Peptide

Preparation of CoFe₂O₄ Nanoparticles with a Biocompatible Polymer Coating and with YSA Peptide Conjugation

The superparamagnetic CoFe₂O₄ nanoparticles were synthesized with a micelle method, and the mean diameter was 8 nm with a size distribution of less than 15%. The detailed experimental procedures are reported in Scarberry, et al., “Magnetic Nanoparticle-Peptide Conjugates for in Vitro and in Vivo Targeting and Extraction of Cancer Cells”, J. Amer. Chem. Soc'y., 130 (31), 10258-10262 (2008).

The nanoparticles (200 mg) and polygalacturonic acid (600 mg, Alfa Aesar) were added into 80 mL of 5 M NaOH solution at ambient temperature. After sonication for 5 h with a Model 60 Sonic Dismembrator (Fisher Scientific), the coated nanoparticles were separated from the solution using a magnet. After being washed a few times with water, the coated nanoparticles were resuspended in distilled water.

Glucuronic acid was also tested as the biocompatible coating with similar procedures. 1.9 mg of peptide having a sequence of GGGGYSAYPDSVPMMSK were added to 10 mL of an aqueous suspension of the nanoparticles with polygalacturonic acid coating (˜1.7×1015 particles/mL). The mixture was sonicated for a few minutes. The solution was protected from light and stored at 4° C. overnight to complete the formation of amide bonds between carboxyl groups on the polymer coating and the primary amine on the C-terminal lysine residue.

The YSA peptide was synthesized using standard Fmoc chemistry as reported in the literature. Clark, et al., J. Biol. Chem., 276: 37431-37435 (2001). A Rhodamine tag was conjugated on the N-terminus, and the four N-terminal glycine residues were used to distance the Rhodamine from the binding region and prevent steric hindrance to receptor binding.

Cell Growth

The BG-1 cell line was provided by Julie M. Hall and Kenneth S. Korach of the Environmental Disease and Medicine Program, Research Triangle Park, NC. The BG-1 cells were cultured in DMEM:F12/50:50 (Invitrogen) supplemented with 10% heat-inactivated fetal bovine serum (FBS) (Invitrogen), 100 U/mL penicillin, and 100 μg/mL streptomycin (Mediatech, Inc., Herndon, Va.) at 37° C., in a 5% CO₂ atmosphere.

The Hey cell line was provided by Gordon Mills, Department of Molecular Therapeutics, The University of Texas, MD Anderson Cancer Center. The Hey cells were propagated in RPMI 1640 (Mediatech) supplemented with 2 mM of L-glutamine (Sigma), penicillin, streptomycin, and 10% heat-inactivated FBS at 37° C., in a 5% CO₂ atmosphere.

Cell Staining

Cells were incubated overnight with 20 mg/mL of fluorescein diacetate (FDA) (Research Organics) or 20 mg/mL 5(6)-carboxyeosin diacetate (Research Organics) at 37° C., 5% CO₂. Cells were removed from the cell culture flask with trypsin+EDTA, washed once with PBS, and resuspended to 2.8×106 cells/mL. For confocal imaging, cells were incubated overnight on chamber slides (Laboratory Tek) and washed the next day with PBS. Rhodamine-labeled nanoparticles with or without conjugated YSA peptide were added to the cells and incubated for 1 h at 37° C., in a 5% CO₂ atmosphere. Cells were washed followed by fixation in 4% paraformaldehyde and coverslipped for imaging analysis.

Mouse Studies

Female nu/nu mice were obtained from Taconic (Hudson, N.Y.) and Balb/c mice were from Harlan (Indianapolis, Ind.). All experiments were conducted with the approval of the Institutional Animal Care and Use Committee at the Georgia Institute of Technology (Atlanta, Ga.).

Microscopy

In vitro studies were conducted using a 40× objective on an Olympus IX71 inverted microscope with green and red filters and a mercury short are HBO lamp. Images and video were taken using an Olympus DP71 12.5 million pixel digital camera. Confocal images were obtained with a 40× objective using a Zeiss LSM 510 laser scanning confocal microscope.

Results and Analysis

The magnetic CoFe₂O₄ nanoparticles were coated with biocompatible polygalacturonic acid to diminish the adverse immune response and also to facilitate the surface modification. After coating of the polymer, the particles became irregular in shape and with a dimension in the range 100-200 nm. Glucuronic acid also worked very well as a biocompatible coating, which formed a shell around each nanoparticle with a thickness of 5-10 nm.

Hey and BG-1 ovarian carcinoma cell lines were used in Example 1. While both Hey and BG-1 lines showed expression of EphA2, the expression was several fold higher in the Hey cell line. For testing the cell targeting and magnetic attraction in vitro, these cancer cell lines were incubated with fluorescein diacetate (FDA) with the green emission at 515 nm, which can be distinguished from the nanoparticulate conjugates with the Rhodamine tag emitting red at 610 nm.

The labeled Hey cells were introduced into a circulating system driven by a peristaltic pump to determine if EphA2 expressing cells could be extracted from a flow stream. A capillary tube, with a flow rate of ˜1.22 mL/min inside, was centered in the circuit and placed above a microscope objective. The continual flow of the green fluorescent Hey cells was observed through the tube.

Approximately 2 min after the introduction of Rhodamine-tagged magnetic nanoparticle—YSA peptide conjugates, a magnet, with a field strength of ˜2600 gauss, was placed on one side of the capillary tube, and the Hey cells accumulated on the tube wall closest to the magnet. When the magnet was removed, the accumulated Hey cell aggregates dispersed rapidly back into the circulating stream.

The cells did not show any response to the magnet if the same magnetic nanoparticles were used but without the YSA peptide ligand.

The capture of the cancer cells by the magnet demonstrated the peptide-functionalized nanoparticles caused the cells to become magnetically attractable.

The specific binding of the YSA-conjugated magnetic nanoparticles to Hey cells was verified by using confocal microscopy studies. Hey cells were incubated in chamber slides and allowed to adhere to the slides overnight. The next day, cells were washed and incubated with Rhodamine-tagged magnetic nanoparticles or with the conjugates of the Rhodamine-tagged magnetic nanoparticle and YSA peptide. Cells were then fixed, and the binding of the magnetic nanoparticles to the cells was examined under fluorescence.

Cells incubated with Rhodamine-tagged nanoparticles showed little or no binding to the particles, while cells incubated with Rhodamine-tagged nanoparticle—YSA peptide conjugates showed binding of the particles over a large amount of the cell surface area (viewed at magnification of 200×). The Hey cells with Rhodamine-tagged nanoparticle—YSA peptide conjugates were also viewed at a higher magnification (400×). The A higher magnification of verified the specific binding of the magnetic nanoparticles to the Hey cells through the YSA peptide/EphA2 interaction and demonstrated that the nonspecific binding of non-YSA-tagged particles approached background levels.

For testing cell capture in vivo, approximately 1.4×10⁶ FDA-loaded Hey cells in 500 μL, of PBS were introduced by injection into the peritoneum of an anesthetized female nu/nu mouse and allowed to disperse for 5 min with gentle abdominal massage to facilitate cell diffusion.

The abdomens of the mice were exposed to 488 nm light under a stereo microscope, and there was no visible fluorescent signal at or around the injection site. This step was followed by the injection of 500 μL of Rhodamine-tagged magnetic nanoparticle—YSA peptide conjugates.

After an additional 5 min of incubation with abdominal massage, the abdomens of the mice were examined under the microscope. No visible fluorescent signal was observed.

After a 2600 gauss magnet with a size of ˜1 cm³ was placed on the skin of the abdomen for 30 s and then removed, the mouse was exposed to 488 nm light again. A green emission from the FDA-loaded Hey cells was clearly visible through the skin at the site of magnet placement, which indicated a large accumulation of Hey cells at this site. When the excitation wavelength was switched to 530 nm to excite the Rhodamine tag, a red emission was visible through the skin at the same spot indicating the existence of Rhodamine-tagged nanoparticulate conjugates, which was consistent with the presence of the dark mass under the bright field.

The magnet was moved over the region and then pulled about 1 cm away from the original aggregation site, and the green and red fluorescent spots shifted to the new location. The lack of any visible fluorescent signal prior to applying a magnet onto the mice suggested the dispersion of cells and nanoconjugates.

The results obtained with a magnet applied indicate that the Hey cells were captured by the magnetic nanoparticulate conjugates in the peritoneal cavity of the mouse via YSA peptide/EphA2 recognition and then were consolidated onto the topside of the cavity by the magnet.

The same study was conducted on FDA-loaded BG-1 ovarian cells. Although the fluorescence of BG-1 cells in vitro was as intense as that of the Hey cells, no visible fluorescent emission through the skin was observed upon exposure of the abdomen of the mouse to the 488 nm excitation light. Fluorescence was visible through the peritoneum once the outer abdominal skin was removed. The emission was much weaker compared to the one produced by the same number of Hey cells. Intense red fluorescence was clearly seen when the excitation wavelength was switched to the 530 nm range, and the nanoparticles were easily seen under a bright field.

This indicated that the lack of BG-1 cell aggregates was not due to a shortage of Rhodamine-tagged magnetic particle conjugates. Thus, the low intensity of the fluorescent emissions from the BG-1 cells could be attributed to a smaller number of cells being sequestered by the nanoparticulate conjugates because of relatively low EphA2 receptor expression by the BG-1 cell line.

A total of four trials were run for each cell line producing similar results.

The difference in extraction efficiencies of the Hey and BG-1 cells implies the specificity of YSA peptide, which was confirmed by in vivo experiments on magnetic extraction of a mixed population of Hey and BG-1 cells within the peritoneal cavity. The Hey cells were incubated with FDA, and the BG-1 cells were incubated with 5(6)-Carboxyeosin diacetate (CDA) with a 560 nm emission. An equal number of cells from each cell line was mixed and introduced into the peritoneal cavity of three Balb/c female mice. After 5 min of cell incubation and abdominal massage, magnetic nanoparticulate conjugates were injected into the peritoneal cavity and incubated for 5 min. The peritoneal fluid was extracted and filtered magnetically before being examined using a hemocytometer to determine the number of green fluorescent (Hey) and red fluorescent (BG-1) cells.

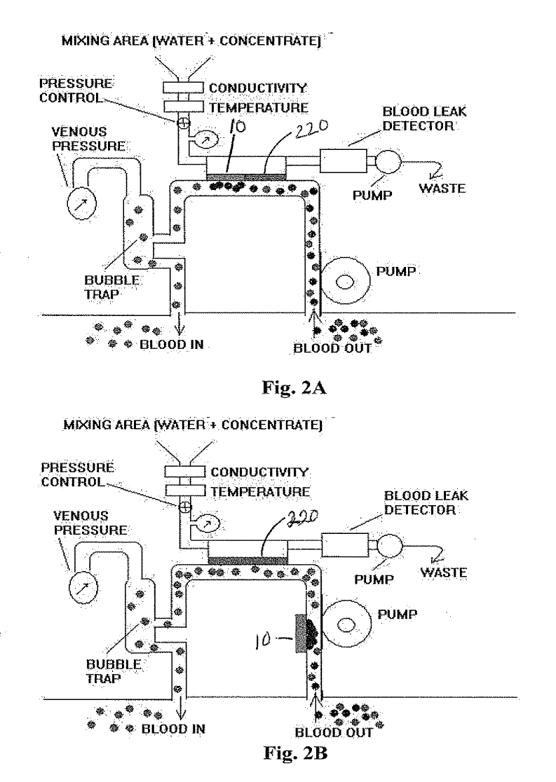

Although the initially mixed cell populations contained 50% Hey and 50% BG-1 cells, Hey cells accounted for 95-100% of extracted cell populations on average from the three trials (see FIG. 3). The scarcity of BG-1 cells in extracted cell populations was consistent with the specificity of YSA peptide—magnetic nanoconjugates. The highly specific binding of the YSA peptide to the EphA2 receptor enabled the magnetic conjugates to differentiate EphA2-rich ovarian carcinoma cells from EphA2-poor cells.

### Example 2

### In Vitro Binding of Superparamagnetic CoFe₂O₄ Nanoparticles Coated with Peptide to Cells in Human Ascites Samples

Nanoparticle Synthesis

The superparamagnetic CoFe₂O₄ nanoparticles were synthesized with a microemulsion technique and the mean diameter was 8 nm with a size distribution of less than 15%. The detailed experimental procedures are reported in Scarberry, et al., “Magnetic Nanoparticle-Peptide Conjugates for in Vitro and in Vivo Targeting and Extraction of Cancer Cells”, J. Amer. Chem. Soc'y., 130 (31), 10258-10262 (2008).

Nanoparticle Coating and Peptide Conjugation

600 mg of CoFe₂O₄ nanoparticles were added to 300 mL of 5M NaOH and sonicated for 10 min (Model 60 Sonic Dismembrator (Fisher Scientific)—power setting of 19). 1800 mg of glucuronic acid was added to the solution and sonication continued for 1.5 hours. The product was magnetically separated using a 5000 gauss magnet, washed 3× in PBS and resuspended in 600 mL of distilled H₂O, bringing the nanoparticle concentration to approximately 1 mg/mL. Particles used in control studies were taken from this stock solution.

To add peptide conjugates, the nanoparticles coated with glucuronic acid were magnetically filtered from 300 mL of the stock solution and resuspended in 30 mL of 0.2 M sodium bicarbonate, pH 9.6. 3 mL from a solution of 0.088 M sodium periodate was added and allowed to react in the dark for 20 minutes. The nanoparticles were again magnetically filtered from solution using a 5000 gauss magnet and resuspended in 30 mL of 0.2 M sodium bicarbonate, pH 9.6. 60 mg of the N-terminally Rhodamine-conjugated peptide GGGGYSAYPDSVPMMSK. (2127.9 g/mol) was dissolved in 6 mL of 0.2 M sodium bicarbonate, pH 9.6, and then pooled with the 30 mL nanoparticle solution. The reaction was allowed to proceed on a platform shaker at room temperature and in reduced light for two hours. 360 μL of sodium cyanoborohydride (5 M) was added to the solution in a fume hood, and the reaction proceeded for an additional 30 minutes under the same conditions. 1.8 mL of 2-aminoethanol was then added to cap unreacted aldehydes and the reaction proceeded for an additional 30 minutes. The final product was magnetically separated from the solution using a 5000 gauss magnet and washed 10× with 0.1 M sodium phosphate buffer and then resuspended in 300 mL of distilled H₂O.

The average diameter of the nanoparticle (approximately 8 nm), the density of the particle (5.29 g/mL), and the mass of the peptide (2127.9 Da) were used to calculate an approximate peptide to particle ratio of nearly 81:1, assuming total reactant consumption.

Peptide Synthesis

The GGGGYSAYPDSVPMMSK peptide was synthesized using standard Fmoc chemistry. See e.g., Clark, et al., J. Biol. Chem., 276: 37431-37435 (2001). A Rhodamine tag was conjugated on the N-terminus, and the four N-terminal glycine residues were used to distance the Rhodamine from the binding region and prevent steric hindrance of EphA2 receptor binding.

Ascites Fluid Preparation

Ascites samples were obtained from the Ovarian Cancer Institute of Atlanta, Ga. Ascites samples were stored at −80° C. in 10% DMSO and thawed in a 37° C. water bath in preparation for use.

Cell Extraction

For each sample being tested, five 12×75 mm round-bottom tubes were labeled as follows: Tube 1—pure ascites, Tube 2—filtrand, Tube 3—filtrate, Tube 4—filtrand control, Tube 5—filtrate control.

500 μL of ascites fluid was added to Tube 1, 2 and 4. 700 μL of ice cold PBS (10% FBS, 1% sodium azide) was added to Tube 1, and it was stored on ice for analysis. 200 μL of peptide conjugated magnetic nanoparticles (1 mg/mL) and 500 μL of ice cold PBS (10% FBS, 1% sodium azide) were added to Tube 2. Tube 2 was vortexed for 15 seconds and shaken for 10 minutes by hand. Tube 2 was then attached to a 5000 gauss magnet for 10 minutes. With the magnet attached to the tube, the fluid was pipetted from Tube 2, placed in Tube 3, and Tube 3 was stored on ice for analysis. The captured magnetic nanoparticles were washed in sterile PBS three times and resuspended in 300 μL of same. The 300 μL nanoparticle solution was filtered through the cap of a 12×75 mm round-bottom tube, the volume was raised to 1200 μL using sterile PBS, and Tube 2 was stored on ice for analysis. 200 μL of magnetic nanoparticles having no peptide conjugate (1 mg/mL) and 500 μL of ice cold PBS (10% FBS, 1% sodium azide) were added to Tube 4. Tube 4 was vortexed 15 seconds, shaken for 10 minutes by hand, and then attached to a 5000 gauss magnet for 10 minutes. While attached to the magnet, the fluid was pipetted from Tube 4, placed in Tube 5, and Tube 5 was stored on ice for analysis. The captured magnetic nanoparticles were washed in sterile PBS three times and resuspended in 300 μL of same. The 300 μL nanoparticle solution was filtered through the cap of a 12×75 mm round-bottom tube, the volume was raised to 1200 μL using sterile PBS, and Tube 4 was stored on ice for analysis. Tubes 1-5 were analyzed immediately using a BD LSR flow cytometer (BD Biosciences).

Cell Surface Staining

300 μL of ascites fluid was resuspended in 12×75 mm round-bottom tubes containing 500 μL of ice cold PBS (10% FBS, 1% sodium azide). All manipulations involving fluorophore-conjugated antibodies were performed in the dark. The samples were centrifuged at 800 RPM and 4° C. for 5 minutes and the supernatant volume was reduced to the 300 μL mark at the base of the tube. 100 μL of ice cold PBS (10% PBS, 1% sodium azide) was added to the sample and the tube was gently agitated to re-suspend the cells. 10 μL of the primary antibody was added and the sample was left to incubate on ice for 30 min. 500 μL of ice cold PBS (10% FBS, 1% sodium azide) was added to each sample and the samples were centrifuged at 800 RPM and 4° C. for 5 minutes.

a. Direct Staining

The previous washing step was performed 3 times and the cells were resuspended in 1200 μL of ice cold PBS (10% FBS, 1% sodium azide) and analyzed immediately using a BD LSR flow cytometer (BD Biosciences).

b. Indirect Staining

The washing step was performed 3 times and the cells were resuspended in 100 μL of ice cold PBS (10% FBS, 1% sodium azide). 10 of the secondary antibody was added and the sample was left to incubate on ice for 30 min. 500 μL of ice cold PBS (10% FBS, 1% sodium azide) was added to each sample and the samples were centrifuged at 800 RPM and 4° C. for 5 minutes. This washing step was performed 3 times and the cells were resuspended in 1200 μL of ice cold PBS (10% FBS, 1% sodium azide) and analyzed immediately using a BD LSR flow cytometer (BD Biosciences).

Flow Cytometry

For each sample analysis using the BD LSR flow cytometer (BD Biosciences) the software configurations (BD Facs/Diva, BD Biosciences) remained consistent. Forward (FSC) and side scatter (SSC) patterns were recorded with a four decade log amplifier. The threshold was set to 2000 and the voltages for the FSC, SSC, FITC, and PE-A parameters were set to 505, 236, 500, and 401 respectively.

For each trial 10,000 events were recorded. Gating of populations was established for a particular patient sample and copied to successive trials to preserve statistical viability.

Flourescence Microscopy

100 μL of the solutions from each tube used for flow cytometry analysis was added to a falcon tube along with 10 μL of Trypan Blue and a small volume was plated for viewing. A 2500 gauss magnet was used to aggregate the magnetic nanoparticles in the solutions from Tube 2 and the aggregate and fluid were placed on a microscope slide for viewing (Olympus X51 Inverted Flourescence Microscope) and imaging (Olympus DP-71).

Results

Flow cytometry was used to analyze pure ascites samples, the filtrand removed from these samples using peptide-conjugated and non-peptide-conjugated superparamagnetic nanoparticles, and the filtrate remaining in the sample after the filtrand was removed.

Bivariate analysis was used to establish significant variation between the number of cells extracted using peptide-conjugated nanoparticles and the number extracted using nanoparticles with no peptide conjugate.

Immunophenotyping techniques were used to verify whether the cells being extracted resided in populations testing positive for markers associated with ovarian adenocarcinoma cells or antigen presenting cells that might be displaying these markers.

Identifying Extracted Cell Populations

A baseline of resident cell populations in ascites samples was determined by observing bivariate displays of forward and side scatter patterns produced when the untreated samples were analyzed using a BD LSR flow cytometer (BD Biosciences). Dot plots for 3 separate trials conducted on patient samples 914 and 923 were prepared. The distributions in the scattergrams displayed morphological consistency across each trial. Quantitative confirmation of this consistency was obtained by gating the visually discreet populations on a density plot of the first trial for each patient copying these gates to each successive trial, and displaying the percent of total recorded events (% Total) for each gated population.

Superparamagnetic CoFe₂O₄ nanoparticles (200 μL−1 mg/mL) coated with glucuronic acid and having N-terminally Rhodamine-conjugated 17 residue peptide functions (GGGGYSAYPDSVPMMSK) were added to 1.0 mL peritoneal effusion samples diluted in sterile PBS (dilution factor 1:2) and given 10 minutes to incubate at ambient temperature. The superparamagnetic nanoparticle conjugates (filtrand) were magnetically filtered from the samples during a 10 minute exposure to a 5000 gauss magnet. The filtrand was washed 3× with sterile PBS, resuspended in 300 μL of same, and filtered through a 12×75 mm falcon tube cap at 800 RPM (4° C.) for 5 minutes. The volume of the filtrand sample was increased to 1200 μL. The filtrate remaining from the extraction was added to a 12×75 mm falcon tube. The filtrand and filtrate were analyzed using the BD LSR flow cytometer (BD Biosciences).

The morphologies of the dot plots for the for flow cytometry analysis of the filtrand and the Citrate samples were dissimilar to the dot plots for flow cytometry analysis (Trials 1-3) of untreated ascites sample from patients 914 and 923. The gates from the untreated ascites trial for each patient were superimposed over the dot plots obtained from the filtrand and filtrate samples to determine the % Total change (delta) for each population.

To reduce background interference caused by aggregates of the coated magnetic nanoparticles alone, 200 μL of the glucuronic acid-coated nanoparticles with and without peptide conjugates were added to separate 12×75 mm Falcon tubes containing 1.0 mL sterile PBS and the samples were analyzed using the BD LSR flow cytometer (BD Biosciences). The bivariate plot produced by the nanoparticles was gated and the gates for populations 2 and 3 of the ascites samples were adjusted to minimize overlap with this region.

The experiment was repeated three times on each patient sample and then conducted three more times using glucuronic acid coated superparamagnetic nanoparticles with no peptide conjugates.

Table 1 compares the average cell counts (Trials 1-3) in the gated populations of untreated ascites samples (Patient 914 & 923) to the average cell counts extracted using nanoparticles with and without peptide conjugates. The “% Total” values for cells in each population expressing EphA2, HEA, CA125, MAC387, and CD83 are also represented. The cell count removed using peptide conjugated nanoparticles for gate P1 appears abnormally high but can be explained by background interference seen in this gate from the nanoparticles alone.

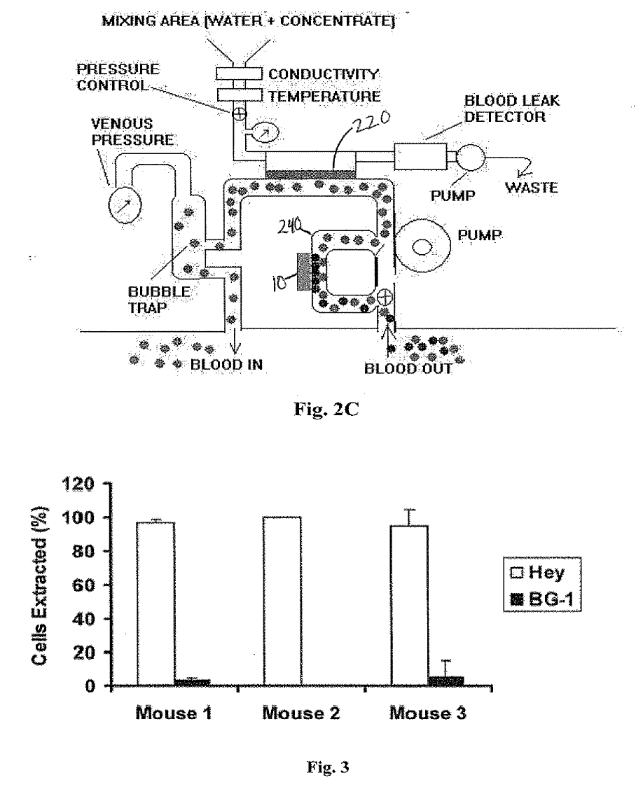

FIGS. 4A-K show the dot plots of flow cytometry analysis for one patient.

TABLE 1

| | | | CELL COUNT | | | | | |
|---|---|---|---|---|---|---|---|---|
| | | CELL COUNT | REMOVED | | | | | |
| | | REMOVED | USING | | | | | |
| | | USING | NANO- | | | | | |
| | CELL COUNT | PEPTIDE | PARTICLES | % TOTAL | % TOTAL | % TOTAL | % TOTAL | % TOTAL |
| | IN | CONJUGATED | WITH NO | SHOWING | SHOWING | SHOWING | SHOWING | SHOWING |
| | UNTREATED | NANO- | PEPTIDE | POSITIVE | POSITIVE | POSITIVE | POSITIVE | POSITIVE |
| | ASCITES | PARTICLES | CONJUGATES | EPHA2 | CA125 | HEA | MAC387 | CD83 |
| | (mean +/− s.d.) | (mean +/− s.d.) | (mean +/− s.d.) | EXPRESSION | EXPRESSION | EXPRESSION | EXPRESSION | EXPRESSION |
| PATIENT | | | | | | | | |
| 914 | | | | | | | | |
| GATES | | | | | | | | |
| P1 | 1570 +/− 266 | 88 +/− 19 | 3 +/− 2 | 0.22% | 0.16% | 0.23% | 0.03% | 0.01% |
| P2 | 5886 +/− 235 | 1058 +/− 161 | 27 +/− 11 | 16.15% | 30.90% | 1.43% | 9.92% | 0.28% |
| P3 | 728 +/− 46 | 654 +/− 144 | 33 +/− 5 | 12.87% | 16.91% | 0.75% | 6.80% | 0.21% |
| P4 | 374 +/− 12 | 18 +/− 2 | 1 +/− 1 | 0.30% | 1.54% | 0.01% | 0.00% | 0.01% |
| PATIENT | | | | | | | | |
| 923 | | | | | | | | |
| GATES | | | | | | | | |
| P1 | 1102 +/− 21 | 151 +/− 11 | 27 +/− 7 | 0.36% | 0.40% | 1.56% | 0.02% | 0.00% |
| P2 | 6132 +/− 117 | 1283 +/− 135 | 308 +/− 27 | 5.6% | 16.45% | 8.06% | 2.03% | 0.61% |
| P3 | 365 +/− 22 | 117 +/− 5 | 24 +/− 3 | 2.71% | 6.3% | 1.52% | 1.02% | 0.11% |
| P4 | 470 +/− 35 | 10 +/− 4 | 0 +/− 1 | 0.58% | 0.73% | 0.11% | 0.02% | 0.07% |

Visual inspection of the plots of Forward Scatter (FSC) versus Side Scatter (SSC) for each patient's untreated ascites samples yielded at least 4 distinct subpopulations and the regions encompassed by gates population 1 (“P1”) and population 1 (“P2”) initially held the largest average number of events (see Table 1). The average % Total values for each population from the filtrand and filtrand control trials are featured in Table 1, as well.

An average of 5.60%+/−1.54% (mean+/−standard deviation), 17.97%+/−2.81%, 89.84%+/−20.58%, and 4.81%+/−0.56% of the cells in the ascites samples from patient 914 were extracted using peptide conjugated nanoparticles from populations P1, P2, P3 and P4 respectively, while 0.19%+/−0.3%, 0.46%+/−0.19%, 4.53%+/−0.74%, and 0.27%+/−0.27% were extracted using nanoparticles with no peptide conjugates from the same respective gated populations.

For patient 923 samples, an average of 13.70%+/−1.0%, 20.92%+/−2.24%, 32.05%+/−2.37%, and 2.13%+/−0.87% of cells were extracted using peptide conjugated nanoparticles and 2.45%+/−0.64%, 5.02%+/−0.45%, 6.58%+/−0.91%, and 0.00% of cells were extracted using nanoparticles with no peptide conjugates from populations P1, P2, P3, and P4 respectively.

Demonstrating the Binding of Ovarian Cancer Cells to Peptide Conjugated Nanoparticles

GGGGYSAYPDSVPMMSK peptide conjugation to nanoparticles using the reductive amination technique described above was verified by taking magnetically aggregated particles that had been cleansed in multiple washes with PBS and viewing them using fluorescence microscopy. An aggregation of magnetic nanoparticles with Rhodamine-conjugated peptides was viewed using an Olympus Rhodamine filter on an Olympus X51 inverted fluorescence microscope. The red fluorescence produced by the aggregate was taken as confirmation that the peptides had been successfully linked.

To visually confirm the adsorption of cells to the nanoparticles subsequent to flow cytometry analysis, a 100 μL sample of the filtrand was stained with 10 μL of Trypan Blue and imaged using bright field microscopy. Having been previously stored at −80° C., it was assumed that the ascites samples would contain dead cell populations. Dead cells can no longer inhibit the passage of Trypan Blue across the cell membrane and would be enhanced visually under bright field imaging using this technique. A 2500 gauss magnet was used to aggregate the magnetic nanoparticles into large clusters to provide further image enhancement. The nanoparticle aggregate appeared blue, suggesting the adherence of dead cells to the nanoparticles.

Demonstrating that Peptide Conjugated Nanoparticles can Successfully Extract Ovarian Cancer Cells from Human Ascites Fluid

Extracted cell counts were compared between experimental and control samples for each patient and trial using Chi-square analysis. The null hypothesis tested was that there was no significant difference between the numbers of cells collected in the experimental and control groups for each gated population. The cell counts from the filtrand obtained using superparamagnetic nanoparticles coated with glucuronic acid and conjugated to a 17 residue peptide sequence (GGGGYSAYPDSVPMMSK) made up the experimental data set (a) and the cell counts from the filtrand obtained using superparamagnetic nanoparticles coated with glucuronic acid and no peptide conjugates made up the control data set (c). The minimum accepted p value of significance was 0.05. If the p value was greater than 0.05, it was concluded that the conjugated peptide had no significant effect on the cell numbers extracted. For p values lower than 0.05, it was concluded that cell counts were significantly enhanced using the peptide conjugate.

The census of each starting population was taken from the flow cytometry analysis of the pure ascites samples used for each trial (H₁ and H₀ for experimental and control groups respectively). The p values for the majority of the patient trials fell well below 0.05; and it was concluded that the superparamagnetic nanoparticles with the 17 residue peptide conjugate were capable of extracting a significantly higher number of cells than particles with no peptide conjugates.

The only trials yielding p values higher than 0.05 came from population 4 of samples analyzed from patient 923. It was determined that any cells extracted from this population were captured non-specifically.

To verify the proclivity of the 17 residue peptide (GGGGYSAYPDSVPMMSK) to target cell populations expressing markers commonly expressed by adenocarcinomas, peritoneal effusions were immunostained using a panel of monoclonal antibodies having a high affinity for those markers.

Single parameter frequency histograms were used to analyze both control and experimental samples. The untreated ascites samples for each patient were used as control specimens, and the autoflourescence levels were recorded using a four decade log amplifier. Experimental samples of peritoneal effusions from each patient were challenged in separate trials with anti-CD83, anti-MAC387, BerEP4, anti-EphA2 and anti-CA125. Each monoclonal antibody had a FITC conjugate, or a secondary antibody with a phycoerythrin conjugate (EphA2).

The histograms from the experimental samples were superimposed over those of the control to determine whether there were any variances between the two. From these graphs, it was shown that 0.22% of the 10,000 events counted for patient 914 samples showed a positive expression for the EphA2 receptor and occurred in gated population P1. The values for P2, P3 and P4 were 16.15%, 12.87%, and 0.30%, respectively. The percentages showing positive EphA2 expression in the samples from patient 923 were 0.36% (gate P1), 5.6% (gate P2), 2.71% (gate P3), and 0.58% (gate P4).

The covariance between the expression levels of EphA2 and CA125 appeared to be positive in gated populations P1, P2, and P3 of samples from patient 914 and gated populations P1, P2, P3, and P4 of samples from patient 923. For instance, CA125 and EphA2 were expressed negligibly in P1 of samples from patient 914 (0.16% and 0.22% respectively) and in greater numbers (30.90% for CA125 and 16.15% for EphA2) in gated population P2.

The highest number of cells expressing CA125, EphA2, HEA, and MAC387 came from gates P2 and P3 for samples from patient 914. However, infrequent HEA expression was seen correspondingly as 1.43% and 0.75% in both these populations.

For samples from patient 923, the number of cells expressing HEA was 8.06% for P2 and 1.52% for P3. There was also a positive covariance between the number of cells expressing EpbA2, HEA, CA125, and MAC387 in P2, P3, and P4 and the highest numbers of cells expressing these markers came from P2.

The expression levels of CD83 were very low for both patient samples. The average CD83+ cell count across all four populations in samples from patient 914 was 0.12%+/−0.14% (mean+/−standard deviation) and 0.19%+/−0.28% for patient 923 suggesting that mature dendritic cells were mostly absent from these ascites samples.

### Example 3

### Ovarian Cancer Study in Mice

An ovarian cancer survival study was conducted to evaluate whether the capture and removal of disseminated tumor cells could be employed as a curative measure to mitigate metastasis and thereby increase longevity. A murine ovarian cancer cell line (ID8 GFP VEGF) transfected with the gene for green fluorescent protein (GFP) and vascular endothelial growth factor (VEGF) expression was used for the study. VEGF expression can expedite tumor progression by stimulating angiogenesis and abating the immune response. The expression of GFP can be analyzed both qualitatively and quantitatively, providing a mechanism for tracking the dissemination of the malignant cells.

The mice used in the study were divided into two control groups and one experimental group. Each group received an intraperitoneal (I.P.) injection of 7 million IDS GFP VEGF cells. The first control group (Control A) contained 7 female C57BL/6 mice (5-8 weeks old), which received no further treatment and was set aside for observation. The second control group (Control B) contained 9 female C57BL/6 mice (5-8 weeks old), which received partial treatment with the extracorporeal device only, without nanoparticles at two and four week time points. The experimental group (Experimental) contained 8 female C57BL/6 mice (5-8 weeks old), which received full treatment with 4 mL, at 10 mg/mL glucuronic coated superparamagnetic nanoparticles at two and four week time points.

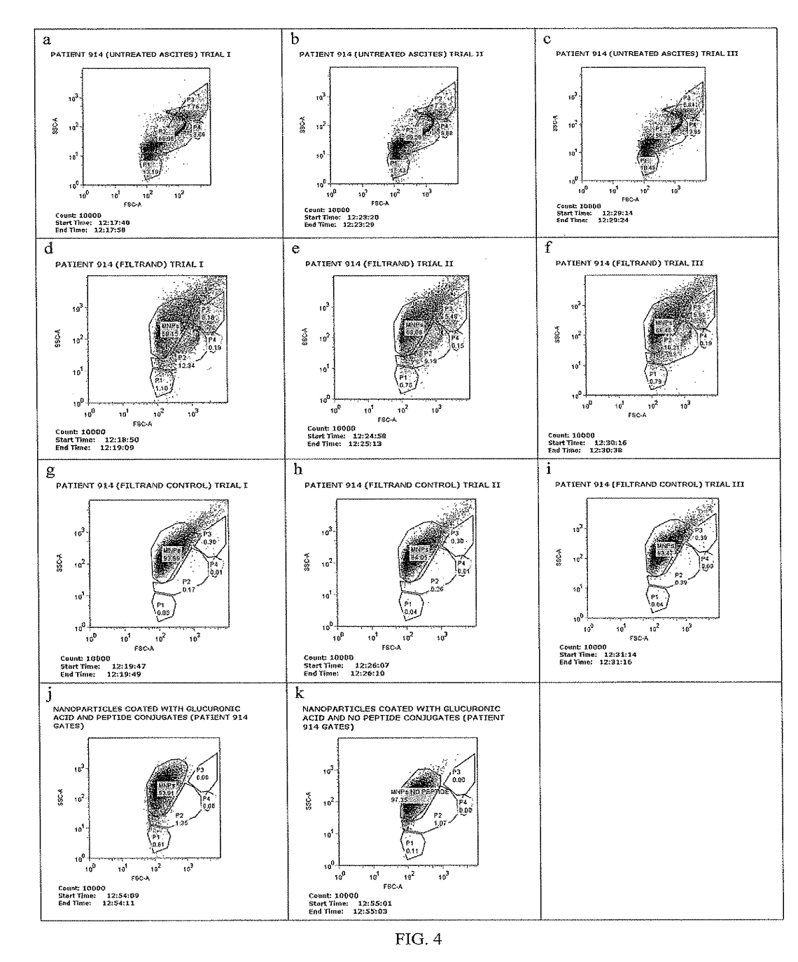

The “full treatment” set up for the Experimental group is illustrated in FIG. 5. It involved cannulating a mouse with an inlet (FIG. 5, Item #6) and outlet (FIG. 5, Item #5) cannula that was used to cycle fluids through the peritoneum.

Prior to cannulation the extracorporeal circuit was primed with sterile PBS or Ringer's Solution. Nanoparticles functionalized with peptides selective for ovarian cancer cell surface receptors were added to a mixing chamber (FIG. 5, Item #3). Once activated, the pump (FIG. 5, Item #4) cycled peritoneal fluids into the mixing chamber. Metastatic cells in the fluids were captured by functionalized magnetic nanoparticles in the mixing chamber and the malignant cell/magnetic nanoparticle conjugates were cycled to a magnetic filtration chamber (FIG. 5, Item #s 1 and 2) where they remained sequestered. The filtered solution (i.e. without the malignant cells and magnetic nanoparticles) was cycled back into the peritoneum of the subject.

Each treatment lasted about 20 minutes, which was the computed time necessary to cycle the average volume of a mouse peritoneal cavity (approx. 2 mL) ten times using the described setup.

Post-operatively, the mouse was disconnected from the extracorporeal circuit and the lines were drained. The drainage was centrifuged to recapture any cells left in the circuit and the pellet was re-injected into the mouse intraperitoneally. This last step was performed to eliminate the possibility of introducing an additional variable into the experiment. By not performing this step the number of metastatic cells in the peritoneum would be effectively decreased by simply drawing them into the circuit which would affect the number of micro metastases.

The same treatment as described was performed on group Control B with one modification. No nanoparticles were used. The peritoneal fluids of this group were only cycled through the circuit to determine whether the device itself had a therapeutic effect on the subject. A qualitative analysis of the magnetic filter contents was performed post-operatively using fluorescence microscopy. A bright field image of the magnetic nanoparticles sequestered by the magnetic filter and a dark field image showing the presence of green fluorescent protein bound to the particles was visible using fluorescence microscopy. These images qualitatively confirmed the capture of metastatic cells from the circuit.

The treatments were performed two and four weeks after the initial I.P. infusion of the 1D8 GFP VEGF cell line. This meant that the malignant cells would have 2 weeks to implant and proliferate within the peritonea of the subjects. This approach increased the likelihood that both the control and experimental groups would eventually develop tumors. Therefore, the treatment was not expected to prevent tumor growth in this model. However, it was expected to retard its spread.

The mice in this study were euthanized once their body mass reached 150% of the mass recorded at the time that they were initially infused with the malignant cells.

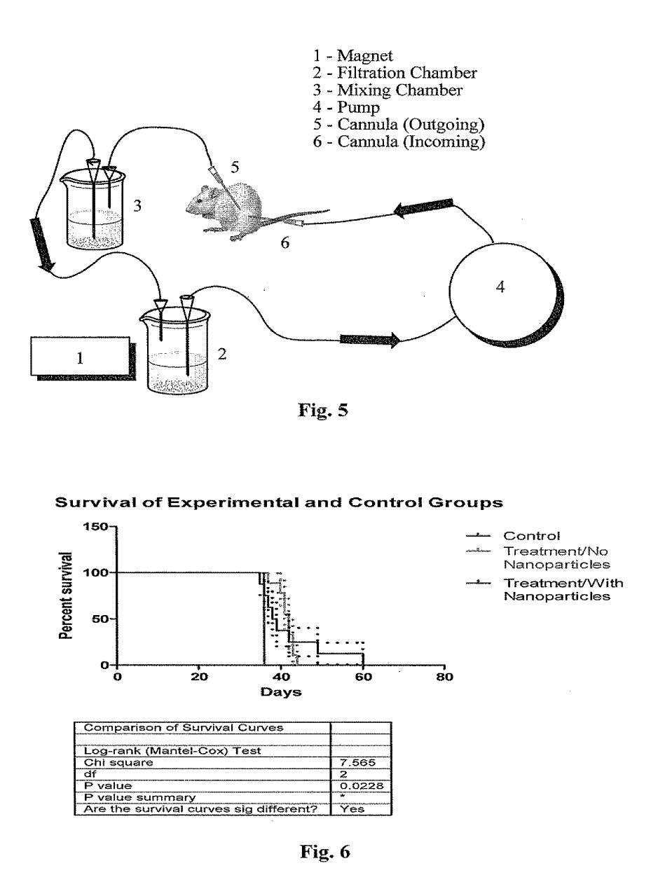

Preliminary studies using the ID8 GFP VEGF cell line had shown that the cells would eventually cause the accumulation of large volumes of ascites in the subject. Using this endpoint, a survival curve was constructed (see FIG. 6). 100% of the subjects in the Control A group had to be euthanized on day 36. Only 11.1% of the subjects from the Control B group were still at risk by day 43 with the final subject being euthanized on day 44. In contrast, 12.5% of the subjects from the Experimental group were still at risk by day 49 with the final subject being euthanized on day 60.

The log-rank (Mantel-Cox) test was used to compare the survival curves of the three groups and a p-value of 0.0228 was obtained providing quantitative support that the Experimental group benefited significantly from the treatment with functionalized superparamagnetic nanoparticles.

### Example 4

### HIV-1 Capture

HIV-1 Samples

Samples of HIV-1 were obtained from the Centers for Disease Control (Atlanta, Ga.).

HIV-1 Detection

Concentrations of p24 protein in RPMI growth medium detected using the ZeptoMetrix HIV-1 p24 Antigen ELISA before and after being challenged with magnetic nanoparticles with and without anti-gp120 conjugates.

Results

The concentration of the HIV-1 virus was significantly reduced in HIV-1 samples media using magnetic nanoparticles with anti-gp120 conjugates. FIG. 6 shows the comparative p24 concentrations of samples containing HIV-1 before and after being challenged with magnetic nanoparticles containing no antibody conjugates and with low and high numbers of magnetic nanoparticles functionalized with anti-gp120.

The experimental conditions and magnetic nanoparticle counts used are featured in Table 2. The means of each group were analyzed using one way ANOVA to determine statistical significance.

TABLE 2
Summary of conditions and results for HIV-1 Capture Study

| CONDITION | VALUE |
|---|---|
| Temperature | 37° C. |
| Incubation Time | 30 min |
| Capture Time (i.e. exposure to magnet) | 10 min |
| # of Magnetic Nanoparticles Introduced | 6.69 × 10¹⁴ |
| (NO Antibody Conjugates) Vial 2 | |
| # of Magnetic Nanoparticles Introduced | 3.52 × 10¹² |
| (WITH Antibody Conjugates) Vial 3 | |
| # of Magnetic Nanoparticles Introduced | 7.04 × 10¹² |
| (WITH Antibody Conjugates) Vial 4 | |
| Viral Particles in Sample | 55,000 |
| Average p24 Concentration in Vial 1 | 53.429 +/− 1.275 (mean +/− |
| (average of 2 trials) pg/mL | standard deviation) |
| Average p24 Concentration in Vial 2 | 36.313 +/− 3.153 (mean +/− |
| (average of 6 trials) pg/mL | standard deviation) |
| Average p24 Concentration in Vial 3 | 38.069 +/− 2.307 (mean +/− |
| (average of 6 trials) pg/mL | standard deviation) |
| Average p24 Concentration in Vial 4 | 16.776 +/− 2.070 (mean +/− |
| (average of 6 trials) pg/mL | standard deviation) |

1. A device for selectively removing a target cell, pathogen, or virus expressing a binding partner on the its surface, the device comprising
an excorporeal circuit, wherein the excorporeal circuit comprises a magnetic filter comprising a magnet capable of generating a magnetic field sufficient to capture magnetic nanomaterials in the magnetic field and a removable, magnetizable substrate capable of capturing magnetic nanomaterials, wherein the magnetizable substrate is a screen; and
a pump in fluid communication with the magnetic filter, wherein the pump moves fluid through the excorporeal circuit.

2. (canceled)

3. The device of claim 1, further comprising a reservoir in fluid communication with the magnetic filter.

4. The device of claim 1, further comprising a mixing chamber between the reservoir and the magnetic filter and in fluid communication with the reservoir and the magnetic filter.

5. The device of claim 1, further comprising a heater for heating fluid moving through the device.

6. The device of claim 1, further comprising a management component in electrical communication or wireless communication with the pump for monitoring or maintaining flow rate of the fluid.

7. The device of claim 1, further comprising a management component in electrical communication or wireless communication with the heater for maintaining the fluid at a predetermined temperature.

8. The device of claim 1, wherein the magnetizable substrate is removable from the magnetic filter.

9. The device of claim 1, wherein the reservoir and magnetizable substrate are sterilizable.

10. The device of claim 1, wherein the reservoir comprises superparamagnetic nanoparticles functionalized with a first binding partner that binds to the binding partner on the surface of the target cell, pathogen or virus.

11. The device of claim 10, wherein the binding partner on the target cell comprises a tumor specific antigen or fragment thereof capable of binding to the first binding partner.

12. The device of claim 11, wherein the first binding partner on the superparamagnetic nanoparticles is selected from the group consisting of nucleic acid aptamers, peptide aptamers, pseudo peptide, synthetic ligands selected for the target, and antibodies or antigen binding fragments thereof.

13. The device of claim 10, wherein the superparamagnetic nanoparticles comprise at least one binding partner per unit surface area of the particles.

14. An ex vivo method for removing a target cell, organism, or virus from a subject in need of treatment, comprising removing a biofluid from the patient and transporting the biofluid into the device of claim 10, passing the mixture of the superparamagnetic nanoparticles and biofluid through the magnetic filter, and either returning the filtrate to the reservoir or the patient, wherein the target cell is a cancer cell.

15. The method of claim 14, further comprising administering replacement fluids to the subject.

16. The method of claim 14, wherein the biofluid is a fluid selected from the group consisting of blood, blood serum, cerebrospinal fluid, lymph, and peritoneal fluid.

17. An ex vivo method for removing a target cell, organism, or virus from a subject in need of treatment, comprising removing a biofluid from the patient and transporting the biofluid into the device of claim 10, passing the mixture of the superparamagnetic nanoparticles and biofluid through the magnetic filter, and either returning the filtrate to the reservoir or the patient, wherein the target cell is an infected cell.

18. The method of claim 17, wherein the infected cell is infected by a virus, bacterium, protozoan, or fungus.

19. The method of claim 17, further comprising administering replacement fluids to the subject.

20. The method of claim 17, wherein the binding partner on the superparamagnetic nanoparticles is selected from the group consisting of nucleic acid aptamers, peptide aptamers, pseudo peptide, and synthetic ligands selected for the target.

21. An in vivo method for removing a target cell organism, or virus from a subject comprising
obtaining a biofluid from the subject;
transporting the biofluid into the device of claim 10,
passing a mixture of the superparamagnetic nanoparticles and biofluid through the magnetic filter, and either returning the filtrate to the reservoir or the patient.

22. The method of claim 14, further comprising sampling the filtrate to determine if superparamagnetic nanoparticles are present therein prior to returning the filtrate to the reservoir or the patient.

23. A ex vivo method for removing nanomaterials having magnetic properties from a subject comprising
obtaining a biofluid containing a nanomaterial having magnetic properties from the subject;
transporting the biofluid into the device claim 1, wherein nanomaterials in the biofluid are captured by the magnetic filter, and either returning the filtrate to the reservoir or the patient.


SEQUENCE LISTING (5 sequences)

SEQ ID NO: 1 - protein, 12 aa, artificial
  synthetic peptide
YSAYPDSVPMMS

SEQ ID NO: 2 - protein, 17 aa, artificial
  synthetic peptide
GGGGYSAYPDSVPMMSK

SEQ ID NO: 3 - protein, 7 aa, artificial
  synthetic peptide
IPVGLIG

SEQ ID NO: 4 - protein, 6 aa, artificial
  synthetic peptide
IVSLRS

SEQ ID NO: 5 - protein, 5 aa, artificial
  synthetic peptide
PQGLA